(19) United States
(12) Patent Application Publication
(10) Pub. No.: US 2003/0060836 A1
(43) Pub. Date: Mar. 27, 2003

(54) Polymer and nerve guide conduits formed thereof

(76) Inventors: Shu Wang, Kent Vale (SG); Andrew C.A. Wan, Baltimore, MD (US); Hanry Yu, Irvine, CA (US); Kam W. Leong, Ellicott City, MD (US)

Correspondence Address: PENNIE & EDMONDS LLP, 1667 K STREET NW, SUITE 1000, WASHINGTON, DC, 20006

(21) Appl. No.: 10/000,297

(22) Filed: Dec. 4, 2001

(30) Foreign Application Priority Data
Dec. 5, 2000 (SG) 200007447-6

(51) Int. Cl. A61B 17/08; C08G 79/02

(52) U.S. Cl. 606/152; 528/400

(57) ABSTRACT

Polymers comprising the subunit

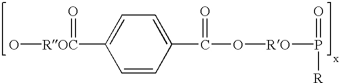

wherein R′ is ethyl or butyl and R and R″ are any suitable side chain or cross link. Nerve guide conduits comprising this and other poly(phosphoesters) are also disclosed.

### 1. FIELD OF THE INVENTION

The present invention relates to a poly(phosphoester) polymer and to nerve guide conduits made from poly(phosphoester) polymers. The present invention also relates to methods of use of such nerve guide conduits and methods of manufacturing polymers.

### 2. BACKGROUND OF THE INVENTION

It is known in the art that nerves with severed axons, but intact somas, may retain the capability of regrowing from the proximal stump to reconnect distally. Current treatments for these defect nerves typically rely on donor tissues obtained from the patient (U.S. Pat. No. 4,662,884). For example, a transected peripheral nerve is rectified clinically by employing an autologous nerve graft, i.e. a segment obtained from a second operative site of the patient and sutured to the two ends of the severed nerve. This has raised the issues of function loss at the donor sites, formation of potentially painful neuromas, structural differences between donor and recipient nerves and shortage of graft material for extensive repair.

The closest alternative to an autologous nerve graft would be an autologous vein or arterial graft [2,3]. The hollow blood vessel acts as a conduit for the regeneration of nerve across the defect, allowing the axons to migrate without obstruction by the degenerating fascicles of the distal stump and preventing the invasion of fibrous scar tissue. However, the use of vein or arterial grafts for ‘entubulation’ is associated with similar limitations as the nerve graft, including a limited supply of the graft and limits to control over the nerve guide dimensions.

To circumvent these problems, synthetic nerve guide conduits (NGCs) have been developed to bridge the nerve gaps by inserting the severed nerve stumps into the two ends of the conduit. The purpose of such nerve guide conduits is to encourage the processes of neuronal growth and regeneration of nerve function. To be effective, these NGCs must be made from materials that meet a wide range of biological and physicochemical prerequisites. The material must be nontoxic, non-carcinogenic, non-antigenic, and must demonstrate favorable mechanical properties such as flexibility, suturability, and amenability to custom fabrication. One example of NGC materials that have been used in combination with sutures is silicones rubber as taught by R. D. Midgley and F. M. Woolhouse, Silicone Rubber Sheathing as an Adjunct to Neural Anastomosis, Surgical Clinic of North America 48, 1149 (1968). Silicone has the disadvantages of being impermeable and non-adsorbable. The use of biodegradable polymers to fabricate NGCs has been the focus for many years. The use of bioresorbable, polyglactin mesh tubing was reported by Molander et al. in Vol. 5, Muscle & Nerve. pp. 54-58 (1982). The use of porous acrylic copolymer tubes in nerve regeneration was disclosed by Uzman et al. in Vol. 9, Journal of Neuroscience Research. pp. 325-338 (1983). Bioresorbable nerve guidance channels of polyesters and other polymers have been reported by Nyilas et al. in Vol. 29, Transactions Am. Soc. Artif. Internal Organs. pp. 307-313 (1983) and in U.S. Pat. No.4,534,349 issued to Barrows in 1985. After accomplishing their function, these guides are supposed to gradually disappear from the host. These biodegradable tubes have the advantage over using biodurable NGCs in dispensing with a second surgery to remove implanted tubes. Such a second operation is often necessary after biodurable NGCs are implanted in order to eliminate the potential problems, such as chronic tissue response and nerve compression, caused by the long-term presence of a rigid tube in the body. In contrast, after a biodegradable tube has accomplished its function, the guide gradually disappears from the host.

Biodegradable polymers differ from non-biodegradable polymers in that they are consumed or biodegraded during therapy. This usually involves breakdown of the polymer to its monomeric subunits, which should be biocompatible with the surrounding tissue. The life of a biodegradable polymer in vivo depends on its molecular weight and degree of cross-linking; the greater the molecular weight and degree of crosslinking, the longer the life. The most highly investigated biodegradable polymers are polylactic acid (PLA), polyglycolic acid (PGA), copolymers of PLA and PGA, polyamides, and copolymers of polyamides and polyesters. PLA, sometimes referred to as polylactide, undergoes hydrolytic de-esterification to lactic acid, a normal product of muscle metabolism. PGA is chemically related to PLA and is commonly used for absorbable surgical sutures, as is PLA/PGA copolymer. However, the use of PGA in sustained-release implants has been limited due to its low solubility in common solvents and subsequent difficulty in fabrication of devices.

Collagen has been one of the most promising materials for construction of NGCs due to its biocompatibility and favourable property of supporting cell attachment and function (U.S. Pat. No. 5,019,087). However, one of the major disadvantages inherent with collagenous materials is their potential antigenicity. Synthetic polymeric biomaterials are potentially advantageous in their unlimited supply and reproducible properties. The versatility of the synthetic system also allows for the fine-tuning of the biodegradation rate, permeability and mechanical properties of the NGC to facilitate peripheral nerve regeneration. Two related patents, U.S. Pat. No. 4,033,938 and U.S. Pat. No. 3,960,152, disclose bioabsorbable polymers of unsymmetrically substituted 1,4-dioxane-2,5-diones which are broadly stated to be useful as tubes or sheets for surgical repair such as nerve and tendon splicing. A similar disclosure in U.S. Pat. No. 4,074,366, col. 6, lines 13-16 and 43-57, relates to poly(N-acetyl-D-glucosamine), i.e. chitin. However, there is no enabling disclosure in the specifications or in their Examples as to how such tubes are to be prepared, the characteristics required, and their method of use.

Other biodegradable polymers of particular interest for medical implantation purposes are homopolymers and copolymers of the alpha-hydroxy carboxylic acids, glycolic acid and lactic acid. These materials undergo hydrolytic scission to form metabolites normal to the body, which are therefore amenable to resorption. A biodegradable polyglactin suture mesh shaped as a tube around a nerve defect to serve as a framework for proliferating cells has been reported in Muscle and Nerve 5, 54-57 (1982). However, less than satisfactory results were achieved in that some of the regenerating axons gained access to the meshes of the polyglactin tube causing the formation of minifascicles. A nerve cuff in the form of a smooth, rigid tube has also been fabricated from a copolymer of lactic and glycolic acids [The Hand 10, (3) 259 (1978)]. U.S. Pat. No. 4,481,353 discloses a bioresorbable polyester terpolymer that also includes an alpha-hydroxy carboxylic acid in conjunction with Krebs cycle dicarboxylic acids and aliphatic diols. These polyesters are useful in fabricating nerve guidance channels as well as other surgical articles such as sutures and ligatures. Regenerated nerves have also been successfully obtained with nerve guides prepared from the homopolymer poly(DL-lactic acid), as measured by myelinated axon counts, for example, in U.S. Pat. No. 5,358,475. Synthetic biodegradable polymers, including polyester urethane [4] and e-caprolactone-lactide copolymer [5-11], have been examined for promoting peripheral nerve regeneration.

One critical issue in regulating nerve regeneration across a gap is the permeability of guide conduits. Permselective conduits with a molecular weight cut-off of 50,000 daltons allowed regeneration of nerves in a mouse sciatic nerve model. The regenerated nerves were characterized by fine epineurium and high numbers of myelinated axons [Aebischer et al., “The Use Of A Semi-Permeable Tube As A Guidance Channel For A Transected Rabbit Optic Nerve”, In Gash & Sladek [Eds] Progress in Brain Research, 78, pp. 599-603 (1988)]. However, problems can arise in fabricating polymer nerve guide conduits with the required permselectivity because other attributes of the polymer, such as viscosity, are also affected by methods known in the art for varying permselectivity.

Despite the identification of various materials which can serve as nerve guide conduits, the results of research to date have revealed significant shortcomings in such prostheses. For example, some of the materials identified above have lead to inflammatory reactions in the test animals and have failed to exclude scar tissue formation within the channels. The total number of axons, the number of myelinated axons, the thickness of the epineurium, and the fascicular organization of nerves regenerated within conduits are all typically less than satisfactory and compare poorly with the original nerve structure of the test animals. Moreover, the loss of sensory or motor function is still the most common outcome of such laboratory experiments. In addition, if the gap distance separating the nerve stumps is too great, regeneration will not occur. There is still demand for developing new biodegradable material with distinct characteristics for nerve guide conduits. It is hypothesized herein that important parameters such as surface properties that affect cell-substrate interaction, porosity and biodegradation that maintains communication with the external environment of the conduits, and delivery of neurotrophic factors that can accelerate axon outgrowth have yet to be optimized.

The class of biodegradable polymers known as poly(phosphoester) (PPE) are known in the art, for example in U.S. Pat. No. 5,256,765 and U.S. Pat. No.6,008,318.

The present invention seeks to alleviate one or more of the above problems.

### 3. SUMMARY OF THE INVENTION

According to one aspect of the present invention, there is provided a polymer comprising the subunit

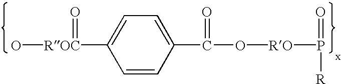

wherein R′ is ethyl or butyl and R and R″ are each any suitable side chain or cross link.

Preferably, R is O(CH₂)ₙCH₃ and n is between 1 and 12.

Conveniently, R is OCH₂CH₃.

Advantageously, R′ is CH₂CH₂.

Conveniently, wherein R″ is (CH₂)ₚ wherein p is between 1 and 12.

Advantageously, R″ is CH₂CH₂.

Preferably, either of both of R and R″ are a functional group and a bioactive component that is conjugated via the functional group.

Advantageously, the bioactive component comprises a neuroactive protein or peptide.

Preferably, the bioactive component comprises a neurotrophic factor.

Conveniently, the neutrophic factor comprises NGF, BDNF, CNTF or FGF.

Advantageously, the bioactive component comprises a cell adhesive peptide.

Preferably, the cell adhesive peptide comprises the amino acids: Arg Gly Asp; Tyr Ile Gly Ser Arg; or Ile Lys Val Ala Val.

Conveniently, either or both of R and R″ are a cross link comprising one or more metal ions.

Preferably, either or both of R and R″ are a cross link comprising one or more organic cross linking agents.

Advantageously, the organic cross linking agent comprises 1,3,5-trihydroxybenzene or (CH₂OH)₄C.

Conveniently, the polymer further comprises the subunit

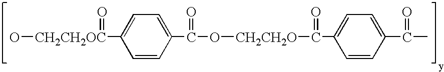

Preferably, x is between 5 and 100 and y is between 5 and 100.

Advantageously, the ratio of x to y is 80:20.

Conveniently, the polymer further comprises the subunit

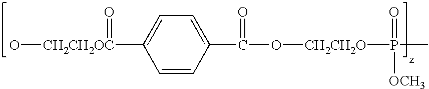

Preferably, the polymer further comprises the subunit

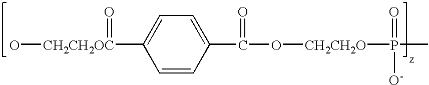

Advantageously, x is between 5 and 100, y is between 5 and 100 and z is between 5 and 100.

In relation to the above statements of the invention, it is to be appreciated that the polymer subunits described do not necessarily comprise single blocks in the polymer backbone. The notations x, y and z indicate the relative number of the respective subunits in the polymer backbone but the units may be included in any order in the backbone, including a random distribution.

According to another aspect of the present invention there is provided a nerve guide conduit comprising a poly(phosphoester) polymer.

According to another aspect of the present invention there is provided a nerve guide conduit comprising a polymer as described above.

Conveniently, the polymer has an average molecular weight of between 10,000 and 25,000.

Preferably, the polymer has an average molecular weight of between 14,900 and 18,900.

Advantageously, the polymer has an average molecular weight of between 15,000 and 17,000.

Conveniently, the conduit has a surface porosity of between 2 and 58%.

Preferably, the conduit has a surface porosity of 35%.

Advantageously, the conduit has a surface porosity of 8%.

Conveniently, the conduit has a diameter of between 1 and 2 mm.

Preferably, the conduit has a diameter of 1.5 mm.

Advantageously, the conduit has a wall thickness of between 150 and 250μm.

Conveniently, the wall thickness is 170 or 240 μm.

Preferably, the conduit comprises a plurality of layers.

Advantageously, the conduit comprises 3 layers.

Conveniently, each layer is between 20 and 30 μm thick.

Preferably, each layer is 25 μm thick.

Advantageously, the outer surface of the conduit has greater microporosity than the luminal surface of the conduit.

Conveniently, the conduit comprises a gene delivery system.

Preferably, the gene delivery system comprises a complex of DNA and a cationic polymer or lipid loaded into the conduit.

Advantageously, the complexes are particles of 20 nm in diameter.

Conveniently, the cationic polymer or lipid comprises polyethylenimine, poly-L-lysine or chitosan.

Advantageously, the cationic polymer or lipid comprises 1,2-dioleoyl phosphatidylethanolamine.

Conveniently, the cationic polymer of lipid comprises Transfast or GenePORTER.

Preferably, the gene encodes a neurotrophic protein or a neuro-active neural fibre growth eliciting molecule.

Advantageously, the gene comprises NGF, BDNF or Bcl-2.

Conveniently, the conduit comprises a sustained protein delivery system.

Preferably, the sustained protein delivery system comprises one or more microspheres, loaded into the conduit and containing a protein such that the protein is released from the microspheres progressively.

Advantageously, the microspheres are made from a polyphosphoester) polymer.

Conveniently, the microspheres are made from a polymer as detailed above. Preferably, the microspheres are made from poly(lactic-co-glycolic acid) or poly(lactide-co-glycolide).

Advantageously, the average diameter of the microspheres is between 5 and 20 μm.

Preferably, the average diameter of the microspheres is 10 μm.

Conveniently, the microspheres release the protein over a period of at least three months.

Advantageously, at least 100 μm of protein is loaded per 10 mm of conduit.

Conveniently, the protein comprises NGF, BDNF, CNTF, epidermal growth factor or fibroblast growth factor.

Preferably, the conduit is loaded with a bioartificial nerve graft comprising Schwann cells.

Advantageously, the conduit is for use in a method of regenerating a severed nerve comprising the step of: inserting each of the respective nerve stumps into the respective first and second ends of the conduit.

According to a further aspect of the present invention there is provided a method of regenerating a severed nerve comprising the steps of: providing a nerve guide conduit as described above, the conduit having first and second ends; and inserting each of the respective nerve stumps into the respective first and second ends of the conduit.

Conveniently, the nerve is in the hand and the conduit is provided adjacent the tendons of the hand.

According to another aspect of the invention, there is provided a method of fabricating a polymer comprising the steps of: providing a solution of the polymer and a solvent; and adding first and second non-solvents to the solution.

Preferably, the solvent has a solubility of greater than 10 mg/ml.

Conveniently, the solvent has a solubility of greater than 100 mg/ml.

Advantageously, either or both of the non-solvents has a solubility of less than 10 mg/ml.

Preferably, either or both of the non-solvents has a solubility of less than 0.1 mg/ml.

Conveniently, one of the solvent and either or both non-solvents has at least 0.5% solubility in the other of the solvent and either or both non-solvents.

Preferably, the method comprises the step of, after adding the first and second non-solvents, increasing the concentration of the second solvent.

Advantageously, the second non-solvent is added to the solution at a concentration of between 0 and 60%(v/v) and is increased to a concentration of between 65 and 75% (v/v).

Conveniently, the concentration of the second non-solvent is increased to 70% (v/v).

Preferably, the method obtains a polymer coating having a predetermined porosity and further comprises the steps of: before adding the first and second solvents, determining the relative concentrations of the first and second solvents required to achieve the predetermined porosity; and adding the first and second solvents in the required concentrations.

Advantageously, the step of determining the required concentrations of the first and second solvents comprises determining the demixing boundary and gelation point of a mixture of the polymer, the solvent and the first and second non-solvents.

Conveniently, the step of determining the demixing boundary comprises titrating pure non-solvent into the homogenous polymer solution and detecting the concentration of non-solvent required to produce permanent turbidity in the solution.

Preferably, the step of determining the gelation point comprises providing a solution of the polymer in the solvent, evaporating the solvent and measuring the rate of change of weight of the solution.

Advantageously, the solvent comprises chloroform.

Conveniently, the first non-solvent comprises water.

Preferably, the second non-solvent comprises methanol.

Advantageously, the polymer is a poly(phosphoester). Conveniently, the polymer is a poly(phosphoester) as described above.

Preferably, the method comprises the steps of: dipping a mandrel into the solution; removing the mandrel from the solution and then drying the polymer solution on the mandrel.

Advantageously, the method comprises the steps of: dipping a mandrel into a solution of the polymer and a solvent; removing the mandrel from the solution and then drying the polymer solution on the mandrel, preferably wherein the solubility of the solvent is greater than 10 mg/ml, more preferably greater than 100 mg/ml.

Conveniently, the method further comprises the step of, after removing the mandrel from the solution, immersing the mandrel in a non-solvent, preferably having a solubility of less than 10 mg/ml, more preferably less than 0.1 mg/ml.

Preferably, the steps of dipping the mandrel into the solution, removing the mandrel and immersing it in a non solvent are repeated one or more times to produce multiple polymer coatings.

Advantageously, the steps are carried out three times.

Conveniently, after immersing the mandrel in the non-solvent, the mandrel is dipped into the solution of polymer before the non-solvent is completely removed from the mandrel.

Preferably, the step of drying comprises freeze drying.

Advantageously, the step of drying comprises vacuum drying.

Conveniently, the method further comprises the step of, after removing the mandrel from the solution, rotating the mandrel horizontally such that variations in the thickness of the coating are reduced.

Preferably, the method further comprises the steps of: casting the polymer in a mold, evaporating the solvent and peeling off the polymer coating from the mold.

Advantageously, the method comprises the steps of: casting a solution of the polymer and a solvent in a mold, evaporating the solvent to form a polymer coating and peeling off the polymer coating from the mold.

Conveniently, the method further comprises the steps of, after peeling the polymer coating from the mold, rolling the polymer around a mandrel and sealing opposing sides of the coating to form a conduit.

Preferably, the sides are sealed by means of a solvent, more preferably chloroform or dimethyl-formamide.

Advantageously, the method comprises the steps of: ad-mixing the protein with a solution or slurry of the poly(phosphoester); and fabricating the conduit from the mixture.

Conveniently, the solution or slurry is aqueous.

In order that the invention may be more readily understood and so that further features thereof may be appreciated, embodiments thereof will now be described, by way of example, with reference to the accompanying drawings.

### 4. BRIEF DESCRIPTION OF THE DRAWINGS

The patent or application file contains at least one drawing executed in color. Contents of this patent or patent application publication with color drawing(s) will be provided by the Office upon request and payment of the necessary fee.

FIG. 1 shows the chemical structure of poly(phosphoesters) used for nerve guidance conduit fabrication according to embodiments of the invention as follows: (a) P(BHET-EOP/TC); (b) P(BHET-EOP-MOP/TC); and (c) P(BHET-EOP-OP⁻/TC).

FIG. 2 shows schematic ternary phase diagrams for P(BHET-EOP)/CHCl₃/H₂O system for: (a) a higher molecular weight polymer; (b) a lower molecular weight polymer.

FIG. 3 shows schematic ternary phase diagrams representing the phase changes that occur during the process of immersion precipitation of: (a) P(BHET-EOP)/CHCl₃/MeOH system; and (b) P(BHET-EOP)/CHCl₃/H₂O system. The cloud point data is assumed to represent the bimodal demixing boundary, considering the relatively long time scale required for the demixing process to occur in this system. The gelation point for the polymer-CHCl₃ system was estimated at 69%(w/w), while the gelation boundary is schematic. The two phase diagrams represent the two extreme cases of immersion precipitation in pure methanol or pure water baths respectively. In the case of water-methanol mixtures, the demixing boundary is expected to shift to intermediate coordinates. This results in changes in the length of time required for the phase separation process to occur, if at all.

FIG. 4 shows a SEM (Scanning Electron Micrograph) of typical appearance of a polymer surface of a vacuum-dried specimen, according to embodiments of the present invention, precipitated in: (a) water; (b) methanol; and (c) 50:50 methanol/water mixture.

FIG. 5 shows a SEM of typical appearance of a polymer surface of a freeze-dried specimen, according to embodiments of the present invention, precipitated in: (a) water; (b) methanol; and (c) 50:50 methanol/water mixture.

FIG. 6 shows a SEM of a poly(phosphoester) nerve guide conduit in accordance with an embodiment of the invention.

FIG. 7 shows a SEM of vacuum-dried polymer surface for specimens, in accordance with embodiments of the present invention, precipitated in immersion baths in which the concentration of methanol in water was incrementally increased from 0 to 70% (v/v) (gradient precipitation). Initial bath methanol concentration (v/v): (a) 0%; (b) 10%; (c) 20%; (d) 30%; (e) 40%; (f) 50%; (g) 60%.

FIG. 8 shows the biocompatibility of P(BHET-EOP/TC) as a NGC material. (A): Rat DRG (Dorsal Root Ganglion) neurons are not affected after treatment with extracts from P(BHET-EOP/TC) conduits extracted for 3 days. NF68 immunostaining in control and treated cell cultures. (B): In vivo Tissue Response to PPE NGC. The tissue capsule at the outer surface of a NGC, 3 months after implantation. The tube would be located at the lower left hand corner of the micrograph and was removed during processing. Hematoxylin and Eosin staining. The original magnification of (A) and (B) was x200 and x400, respectively.

FIG. 9 shows the results of experiments measuring the swelling and wall thickness changes of nerve guide conduits in accordance with embodiments of the present invention over time. In vitro swelling of P(BHET-EOP/TC) conduits. (A) Weight increase. N=7 in Type I and 8 in Type II conduits. The data is present as mean ±SEM.*: significant difference between Type I and Type II conduits, p<0.05. (B) Increase in wall thickness. N=6 in each type of conduit.

FIG. 10 shows the results of experiments into weight loss and decrease of weight-average molecular weight of PPE NGCs, in accordance with an embodiment of the invention, in a time-dependent manner under physiological conditions. Thus the experiments show the in vitro degradation of P(BHET-EOP/TC) conduits. (a) Percentage weight loss. N=7 in Type I and 8 in Type II conduits. (b) Percentage decreases in weight-average molecular weight. At least three conduits were used for sample preparation for a GPC analysis at each point.

FIG. 11 shows in vivo degradation of P(BHET-EOP/TC) conduits, in accordance with embodiments of the present invention. SEM images (A) and (D) are from Type I and II NGCs, respectively, before implantation. SEM image (B) displays the surface change in Type I conduits 6 weeks after implantation and (E), (F) show changes in Type II conduits at 3 months. (C) is the macroscopical appearance of a Type II conduit at 3 months.

FIG. 12 shows macroscopic views of (A) the sciatic nerve repaired at the proximal stump with a Type II PPE conduit, in accordance with an embodiment of the present invention, pre-filled with saline at the time of implantation. (B) A macroscopic picture of the matrix cable across 10-mm gap at 3 days in a Type II PPE conduit, in accordance with an embodiment of the present invention. (C) Micrograph of a longitudinal section of the acellular fibrin matrix found at 3 days. The original magnification was ×200. (D) A regenerated nerve 3 months after surgery. A conduit, in accordance with an embodiment of the present invention, and 14 mm in total length, was used to bridge a 10-mm gap.

FIG. 13 shows NF68 immunoreactive axons in Type II PPE conduits, in accordance with an embodiment of the present invention, 3 months after implantation. (A) At the proximal stump; (B) At the distal stump; (C) Comparison of the NF68 immunoreactivity expressed in arbitrary units.

FIG. 14 shows PEI-mediated Gene Expression in the Rat DRG after Gene Delivery in Nerve Guide Conduits (PEI/pcDNA3 lacZ, 4 μg) in accordance with an embodiment of the present invention. 20 μl of PEI/pcDNA3lacZ complexes were loaded into a NGC conduit after suturing the conduit with the transected rat sciatic nerve. The dorsal root ganglions were collected and sectioned for b-Gal staining.(A) day 0; (B) day 1; and (C) day 2.

FIG. 15(C) shows PEI-mediated Gene Expression in the spinal cord 2 days after gene delivery in NGC(PEI/pcDNA3 lacZ, 4 μg). 20 μl of PEI/pcDNA3lacZ complexes were loaded into a NGC conduit, in accordance with an embodiment of the present invention, after suturing the conduit with a transected rat sciatic nerve. The spinal cord were collected and sectioned for b-Gal staining. FIG. 15(A) shows a control having only PEI. FIG. 15(B) shows a control having only DNA.

FIG. 16(B) shows regenerated cables collected one month after PEI/pcDNA/BDNF complexes (4 μg in 20 μl ) were loaded into a NGC conduit, in accordance with an embodiment of the present invention, after suturing the conduit with a transected rat sciatic nerve. FIG. 16(A) shows a control. The results show that PEI-mediated BDNF expression promotes nerve regeneration.

FIG. 17(B) shows the histological examination of regenerated cables collected one month after PEI/pcDNA/BDNF complexes (4 mg in 20 ml) were loaded into a NGC conduit, in accordance with an embodiment of the present invention, after suturing the conduit with a transected rat sciatic nerve. FIG. 17(A) is a control. Micrographs of semi-thin sections show myelinated axons. The higher fiber density in BDNF loaded NGC is to be noted. Toluidine blue staining. The original magnification ×1000. The figures show that PEI-mediated BDNF expression promotes nerve regeneration.

FIG. 18 shows western blot analysis of human Bcl-2 expression to compare gene transfer efficiency of different DNA carriers. Rat brainstem samples were collected 2 days after tongue injection. Lane 1 and 10: size marker; lane 2: positive control, Bcl-2 transfected NT2 cells; lane 3: injected with chitosan/Bcl-2 complex (0.05% chitosan in HAC); lane 4: injected with Poly-L-lysine/Bcl-2 complex (100 ng/ul of PLL in DMEM); lane 5: injected with PEI/Bcl-2 complex (N/P=10/1); lane 6: injected with PEI solution alone; lane 7: injected with naked Bcl-2 plasmid DNA; lane 8: Bcl-2/Transfast complex; lane 9: Bcl-2/GenePORTER complex. Molecular weights of protein standards are stated on the left site of the blot. Tissue extracts with 20 μg of total proteins per lane were electrophoretically separated in 12.5% SDS-PAGE, transferred to a nitrocellulose sheet, and inimunoreacted with a monoclonal antibody against human BCL-2.

FIG. 19(A) depicts microspheres fabricated with 1% PVA. FIG. 19(B) depicts microspheres fabricated with 10% PVA. This shows that a high concentration of PVA is required to fabricate microspheres with a small diameter.

FIG. 20 shows microsphere distribution within a regenerated tissue cable after loaded into a NGC, in accordance with an embodiment of the present invention. FITC-BSA was used to prepare microspheres. The regenerated tissue cable was collected one week after tube implantation and microsphere loading. The distribution was examined after sectioning of the tissue cable.

FIG. 21 shows the results of experiments into the level of entrapment of protein within microspheres. The percentage of protein encapsulated in each of the polymeric microspheres is shown. The amount of protein was recovered after dissolving microspheres in chloroform and extracting the protein in water.

FIG. 22 shows release profile of BSA protein from biodegradable polymeric microspheres. The cumulative protein release profile was normalized to the initial amount encapsulated.

FIG. 23 shows release profile of NGF from microspheres made from PPE polymer p(DAPE-EOP). The cumulative values were normalized to the initial amount of NGF encapsulated.

FIG. 24 shows nerve regeneration within conduits loaded with NGF microspheres. A: Immunostaining of 68 kD neurofilament protein, a specific neuronal cytoskeleton marker, 2 weeks after implantation of the NGC, in accordance with an embodiment of the present invention, that was filled with a solution of PPE microspheres containing 40 ng of nerve growth factor (NGF). B: control. PPE microspheres without NGF.

### 5. DETAILED DESCRIPTION OF THE INVENTION

Definitions:

| | |
|---|---|
| BDNF | brain derived neurotrophic factor |
| BHET | bis(hydroxyethyl)-terephthalate |
| Bioactive Component | a neutrotrophic factor or cell adhesive peptide |
| CNTF | ciliary neurotrophic factor |
| EOP | ethyl phosphorodichloride |
| FGF | fibroblast growth factor |
| GenePORTER | cationic lipid comprising 1,2-dioleoyl |
| | phosphatidylethanolamine |
| MOP | methyl phosphorodichloridate |
| NGC | nerve guide conduit |
| NGF | nerve growth factor |
| PI | polydispersity index |
| Poly(phosphoester) | a polymer having a subunit of the formula |
| | 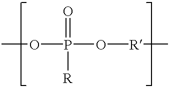 |
| PPE | poly(phosphoester) |
| TC | terephthaloyl chloride |
| Transfast | ±N,N[bis(2-hydroxy- |
| | ethyl)-N-methyl-N-[2,3-di(tet- |
| | radecanoyloxy)propyl] ammonium |
| | iodide/1,2-dioleoyl phosphatidylethanolamine. |

Embodiments of the invention provide biodegradable tubular devices useful as guide conduits for severed regenerating nerves. Such a conduit can be used to repair nerve defects on the face or upper and lower extremities caused by injury, operation or other factors that result in permanent loss of sensation and motor functions. With the severed nerve stumps inserted into the two ends of the tubes, the conduits may provide directional guidance for nerve outgrowth, prevent invasion of scar tissue, maintain endogenous trophic or growth factors, and repel external factors that are inhibitory to nerve outgrowth. The advantages of such conduits instead of using donor tissues from patients for treatments of nerve defects include: preserving function at the potential donor sites, eliminating the risk of formation of painful neuromas at the donor sites and reducing the number of surgical procedure involved.

The devices are formed using a biodegradable poly(phosphoester) matrix. With this polymer, it is feasible to produce a device having a surface porosity ranging from 2 to 58% and a molecular weight cut-off of up to 12000 kD. The device, through its transient in vivo presence, provides an effective aid that persists for a period of time sufficient to promote nerve regeneration, and essentially lacks host toxicity upon degradation. In some embodiments, the device is incorporated with sustained neuronal trophic factor release systems and gene delivery systems, thus providing enhanced promotion of severed nerve regeneration.

Embodiments of the invention include a class of poly(phosphoester) polymers that eliminate or substantially reduce many of the problems and disadvantages associated with the prior art attempts at fabricating nerve guide conduit devices. In particular, by using novel processing methods that are effective in controlling porosity, a nerve guide conduit made from the poly(phosphoester) exhibits good performance in promoting nerve regeneration, high biocompatibility, adjustable biodegradability, negligible swelling, no crystallization after implantation and the potential of coupling neuroactive biomolecules.

The poly(phosphoester) class of biodegradable polymers has structural versatility and attractive physico-chemical properties. The versatility of this class of polymers comes from the versatility of the phosphorus atom, which is known for its multiplicity of reactions. The potential of conjugating bioactive components such as neurotrophic factors or ligands to a poly(phosphoester) to enhance nerve regeneration provides a distinct advantage in the application of this polymer as a nerve guide material. Modification of the surface chemistry within the lumen of a NGC in this way may help to direct nerve regeneration and/or to support nerve fiber maturation. Furthermore, the P(BHET-EOP/TC) type of poly(phosphoester) possesses excellent film-forming properties and processability.

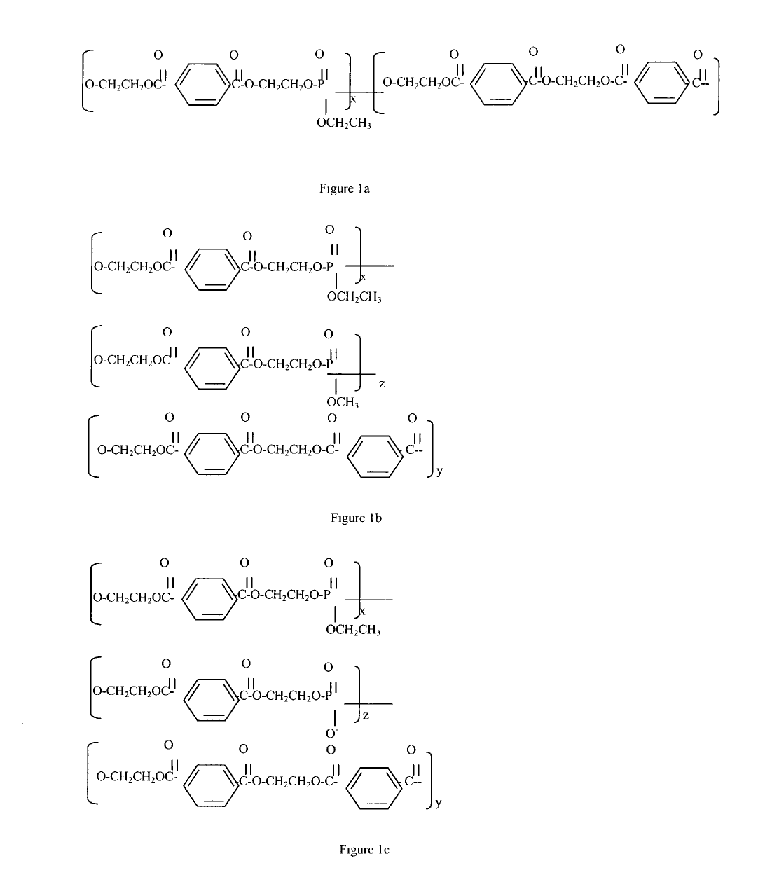

The phosphoester bond in the PPE backbone can be cleaved by water under physiological conditions. The rate of biodegradation of the poly(phosphoester) compositions of the invention may also be controlled by varying the hydrophobicity of the polymer as described in U.S. Pat. No. 5,256,765. When the composition of the polymer is used for long term delivery of a therapeutic agent a relatively hydrophobic backbone matrix, for example, containing bisphenol A, is preferred. It is possible to enhance the degradation rate of the poly(phosphoester) or shorten the functional life of the device, by introducing hydrophilic or polar groups, into the backbone matrix. Further, the introduction of methylene groups into the backbone matrix will usually increase the flexibility of the backbone and decrease the crystallinity of the polymer. Conversely, to obtain a more rigid backbone matrix, for example, when used orthopedically, an aromatic structure, such as a diphenyl group, can be incorporated into the matrix. Also, the poly(phosphoesters) can be crosslinked, for example, using 1,3,5-trihydroxybenzene or (CH₂OH)₄C, to enhance the modulus of the polymer. Similar considerations hold for the structure of the side chain (R) of FIG. 1.

The ultimate hydrolytic breakdown products of the polymers are phosphate, alcohol and diol. By rational design of the building blocks, PPE polymers are biocompatible. In view of their intended function as a therapeutic agent-bearing implant or prosthesis to be introduced into a subject in vivo, it is desirable that these compositions be essentially non-inflammatory, and non-immunogenic.

An advantage of PPE polymers is the availability of functional side groups which allow the chemical linkage of therapeutic agents to the polymers. For example, drugs with carboxyl groups can be coupled to the phosphorous atom via an ester bond, which is hydrolyzable. The rate of therapeutic agent release will then be dependent on the hydrolytic cleavage of the polymer therapeutic agent conjugate. This pendant delivery system has the advantage of attaining a high drug loading level. Therapeutic agents which exist in the liquid state can also be accommodated. Alternatively, therapeutic agents containing two hydroxyl groups can be directly incorporated into the backbone of the polymers. For instance, steroids such as estradiol can be reacted with dichlorophosphates to form the polymer. Other therapeutic agents can also be derivatized for incorporation into the backbone. For instance, a drug with two amino groups can be reacted with the carboxyl group of a hydroxyl carboxylic acid. The hydroxyl groups can then be used to form the poly(phosphate ester). A sustained delivery is then effected by hydrolysis of the polymeric prodrug.

The combination of dip-coating and immersion precipitation technology has been employed in embodiments of the present invention for the fabrication of the nerve guide conduit devices from poly(phosphoester) with an ethylene terephthalate backbone, P(BHET-EOP/TC). Two routes have been established for the generation of porosity, that is immersion precipitation in water followed by freeze-drying, or the use of a non-solvent pair to effect demixing and gelation respectively, and the effects of these parameters have been rationalized by employing ternary phase diagrams. The latter approach can be seen as a new concept for the formation of porosity in this system, i.e. the use of a combination of two non-solvent types; the first, a good ‘liquid-liquid demixer’ to achieve phase separation and the second, a good ‘gelator’ to capture the phase separated structure. Alternatively, the resulting porosities can also be considered to be due to a shift in the coordinates of the demixing boundary.

As such, the porosity of P(BHET-EOP/TC) coatings can be controlled by employing different initial bath compositions with respect to methanol-water ratio, and then gradually increasing the concentration of methanol to achieve gelation. The availability of processing methods which afford good porosity control will be of ever increasing importance in the fabrication of new generation nerve guidance conduits, where permeability characteristics remain a prime attribute which defines their interaction with the biological environment.

Permselective conduits may support regeneration by allowing inward passage of nutrients and growth or trophic factors from the external host environment, while preventing the inward migration of scar-forming cells. Cells participating in the wound healing phenomena are known to release various peptide growth factors. Several of these factors have molecular weights in the range 10-40,000 daltons. For example, activated macrophages secrete numerous growth factors, including NGF, bFGF, and apolipoprotein E. Schwann cells distal to the nerve injury express low affinity NGF receptors, as well as apolipoprotein B and E receptors. Binding of apolipoprotein E to these receptors may enhance lipid uptake which can eventually be used in remyelination. (Aebischer et al., Brain Research, 454, pp. 179-87 (1988)). Appropriate choice of the molecular weight cut-off for the permselective conduits will allow retention of laminin (a high molecular weight glycoprotein) within the nerve guidance channel. Similarly, blood vessels located in the proximal nerve stump may supply high molecular weight serum molecules such as fibronectin or glycoproteins that have supported neuronal survival and neurite extension in vitro (Aebischer et al., Brain Research, 454, pp. 179-87 (1988)). Accordingly, embodiments of the present invention provide an effective way to control porosity during fabrication of nerve guide conduits.

Coatings of varying porosities are obtained by immersing mandrels coated with a solution of the polymer in chloroform into non-solvent immersion baths, followed by freeze or vacuum-drying. The porosity decreases with an increase in polymer molecular weight, drying time before precipitation and concentration of polymer solution. Nerve guidance conduits are fabricated by the application of multiple coating layers. Conduits made from the polymer of M_{w} 14900 (Surface porosity of 35%) possesses a molecular weight cut-off of between 4400 and 12000 towards FITC-dextran, and the permeation constant towards FITC-dextran of M_{w} 4400 was 2.8×10⁻⁹ cm² s⁻¹. To afford improved porosity control, a new system was developed which employs the contrasting phase separation behavior of P(BHET-EOP/TC)/CHCl₃ solution in methanol and water. Mixtures of these two non-solvents allow the demixing boundary to be shifted accordingly to achieve surface porosities ranging between 2% to 58%.

The conduits made with the above methods were non-toxic to all six types of cells tested, including primary neurones and neuronally differentiated PC12 cells, and produced a negligible inflammatory response in the surrounding tissue after in situ implantation in the sciatic nerve of the rat. In the rat sciatic nerve model with a 10 mm gap, the regeneration of peripheral nerves was observed, with a high number of myelinated axons close to normal values. The conduits were degradable in vivo, as evidenced by fragmentation, increased roughness on the tube surface and a mass decrease in a time-dependant manner. The MW (molecular weight) of the polymers dropped 33% within 3 months. Furthermore, the conduits showed negligible swelling and no crystallization after implantation.

Embodiments of the invention also include loading the nerve guide conduits with polyethylenimine/DNA complexes which improves the regeneration quality of severed nerves. The complexes migrate by retrograde axonal transport to neuronal cell bodies in the spinal cord after being internalized by nerve terminals. Gene expression is detectable as early as 24 hours after loading and lasts for at least 2 weeks. Several other cationic polymers or lipids, such as poly-L-lysine, chitosan, Transfast or GenePORTER, may deliver genes through the same way. The gene delivery method of embodiments of the invention bypasses the blood-brain barrier and provides a practical therapeutic strategy that is non-invasive to the spinal cord.

Using microspheres containing neuron trophic factors aside from polyethylenimine/DNA complexes within the conduits improves the regeneration quality of severed nerves as well. The microspheres are composed of biodegradable poly(phosphoester) polymers and are loaded with the nerve growth factor (NGF). A sustained release of NGF proteins from the microspheres for at lease three months was observed as the polymer degrades under a physiological condition.

A more detailed description of the preferred embodiments of the invention follows.

Conduit Fabrication:

The primary constituent of the nerve guidance conduit is a poly(phosphoester) with an ethylene terephthalate backbone, namely poly (bis(hydroxyethyl) terephthalate-ethyl ortho-phosphate/terephthaloyl chloride), P(BHET-EOP/TC) (FIG. 1a) where the ratio of BHET-EOP (x) to TC (y) groups in the backbone can be varied to fine-tune the physical properties of the polymer. In another embodiment of the invention, the poly(phosphoester) has the structural formula shown in FIG. 1b, which can be demethylated before or after NGC fabrication to yield the poly(phosphoester) shown in FIG 1c. These two polymers can be represented by the abbreviations P(BHET-EOP-MOP/TC) and P(BHET-EOP—OP⁻/TC) respectively, where MOP represents methyl ortho-phosphate and OP⁻ represents the free phosphate group. Again, the ratios of the x, y and z components can be varied to achieve the desired properties for the NGC. In preparing the NGC of the present invention, the above-mentioned polyphosphoesters were first synthesized by a reported method. (H-Q Mao et al., 1999)

When a polymer coating comes into contact with a non-solvent (in this case, water), influx of the non-solvent results in a process of phase separation, whereby the polymer solution separates into a polymer-rich and polymer-lean phase. When the coating is allowed to stand in water for some time, gelation of the polymer rich phase occurs, and this results in ‘freezing’ of the 2 phase structure. The process of freeze-drying allows removal of water from the polymer-lean phase without significant shrinkage occurring, and the result is a porous poly(phosphoester) coating. In contrast, vacuum-drying of the coating results in shrinkage and collapse of the pores due to the surface tension of the evaporating liquid. This results in a dense, non-porous coating. While two extremes of surface porosity can be achieved by using either freeze-drying or vacuum drying, a finer control of the porosity can be achieved by a process of ‘gradient precipitation’, which is one aspect of the present invention.

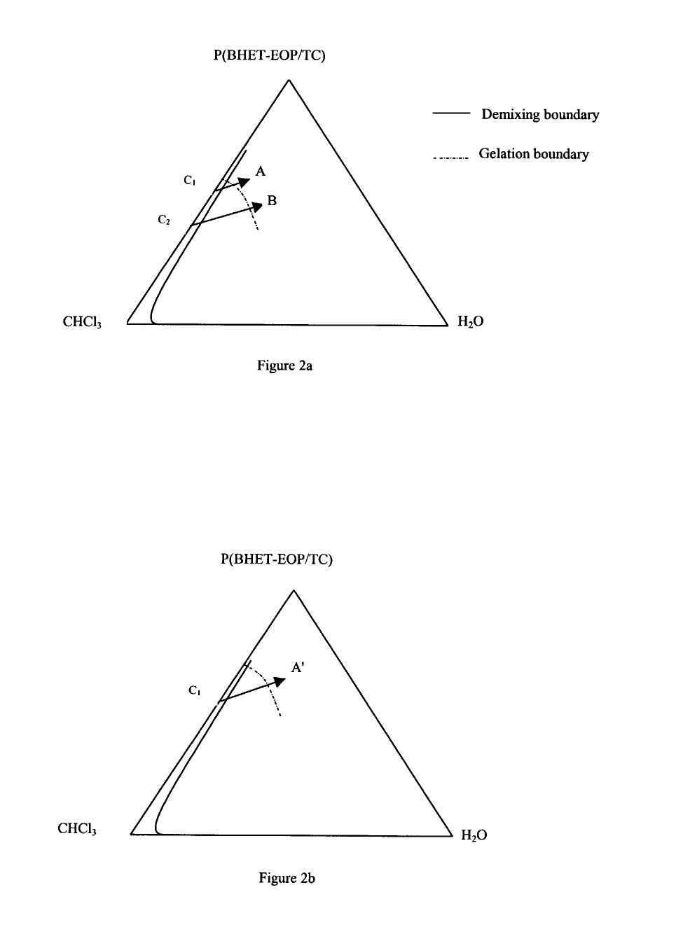

The path of the immerse precipitated coating can be schematically illustrated in the ternary phase diagrams of FIGS. 2a and 2b. The influence of different solution concentrations and molecular weight of poly(phosphoester) can be explained by reference to these diagrams. The gelation boundary for the polymer with the higher molecular weight is expected to be lower than that of the lower molecular weight polymer. Thus the polymer of higher molecular weight would require a lower solution concentration in order to achieve gelation. Taking the same concentration of polymer (c1) for both systems, it can be seen that the path of the system through the phase separated region is shorter for the higher molecular polymer (c₁-A) whereas it is longer for the lower molecular weight polymer (c₁-A′). This means that a shorter time is available for the higher molecular weight polymer to undergo solution demixing before gelation occurs, resulting in smaller pores and a lower pore density. Experimentally, there is a significant difference in porosity for polymer coatings processed in the same way for polymers of different molecular weight.

Taking the case of the higher molecular weight polymer at two different concentrations (c1 and c2) (see FIG. 2a), it is clear that a lower concentration of polymer solution (c2) is expected to undergo a larger degree of phase separation, compared with the higher concentration (c₁) and this leads to a higher porosity for the former. This is reflected in the results of an experiment investigating the influence of polymer solution concentration on porosity, where surfaces of coatings obtained from higher concentrations of polymer were less porous.

The effect of drying time of the polymer coating before precipitation in the non-solvent bath is in principle due to the effect of concentration. At longer drying times, the evaporation of chloroform from the coating naturally leads to higher, albeit unknown concentrations of polymer in the coating, resulting in lower porosities. This is reflected in the surface porosities of the polymer coatings obtained at different drying times.

However, porosity is not the only consideration when selecting the optimal concentration of polymer solution to be used for conduit fabrication. Concentration exerts a profound effect on solution viscosity and this in turn affects the quality of the coating in several ways. Firstly, viscosity affects the leveling phenomenon of coatings. During the application of coatings, imperfections such as waves and furrows appear on the surface. To obtain good coating quality, these imperfections must disappear before the wet coating solidifies. Surface tension is the main driving force of the leveling process, while viscosity is the main force that resists it. Therefore, to facilitate leveling, it is desirable to employ coatings of low viscosity. Low viscosity coatings however, cannot always be used as sufficiently thick coatings would then be difficult to deposit. Furthermore, when the coating is deposited on a vertical surface (as in the case of dip-coating), a high viscosity is required to prevent sagging, i.e. downward flow of the coating fluid under the influence of gravity. Based on these considerations, an optimal viscosity from a chosen solution concentration would have to be employed to obtain an acceptable coating.

During preliminary experiments, it was discovered that the polyphosphoester) solution exhibited a thixotrophic property. This means that the solution undergoes a drop in viscosity as force is applied; but as shear forces are reduced, viscosity increases at a lesser rate to produce a hysterisis loop. This thixotrophic property of the solution could be put to good use during the process of conduit fabrication. Due to the high contact angle of the solution on the PTFE mandrel surface (i.e. poor wetting), application of a force to the liquid was necessary to enable it to spread and wet the mandrel sufficiently. This was achieved by drawing the mandrel out of the dip-coating solution at a high rate. The shear force generated by this procedure also served to lower the viscosity of the coating, enabling leveling to proceed efficiently. In this way, a substantially thick, yet uniform first coating could be applied onto the mandrel surface.

The solution's thixotrophic property also meant that it would develop a viscous and gradually gel-like state if allowed to stand over several days. This could be alleviated by continuous overnight agitation of the solution in a shaker, before use on the following day.

In order to obtain a polymer tube with the requisite integrity and mechanical properties for application, multiple coating steps (≧3) are required. To ensure that the porous structure of a coating is not disrupted by application of the subsequent coating, it is essential that the non-solvent (water) is not completely removed during the horizontal drying process between successive coatings. Using an optimized procedure, multiple layered conduits have been made from both Batch 1 and Batch 2 polymers and implanted them in a rat model to investigate peripheral nerve regeneration [24]. It was shown that these conduits served the function of entubulation, and facilitated nerve regeneration.

Control of Porosity:

The permeability of nerve conduits is an important issue, as demonstrated by previous studies. It determines the inclusion or exclusion of factors that affect nerve regeneration. At the morphological level, both the extent of permeability (molecular weight cut-off) and rate of permeation is reflected by the porosity of the material. Furthermore, porosity also affects the susceptibility of the polymeric material towards in vivo swelling and degradation.

Polymer molecular weight, the drying time of the polymer coating before precipitation and concentration of the coating solution are parameters that influence the porosity of the polymer tubes. However, there are drawbacks in employing these parameters for the purpose of porosity control. Firstly, it would not be feasible to use a different molecular weight of polymer every time a new degree of porosity were required. Secondly, using drying time as a method of porosity control suffers from the fact that rapid vaporization of chloroform from the coating results in gelation of the surface within several minutes of drying. This leads to minimal variation in porosity whether the coating is air-dried for 5 minutes or 30 minutes. In addition, uneven drying leads to a heterogenous surface. Concentration of coating solution is not a good parameter to vary for porosity control as concentration affects not only porosity, but viscosity of the polymer solution as well. A uniform and adequately thick polymer coating can only be achieved within a narrow range of viscosity, dictated by considerations of coating phenomena such as wetting, leveling and sagging.

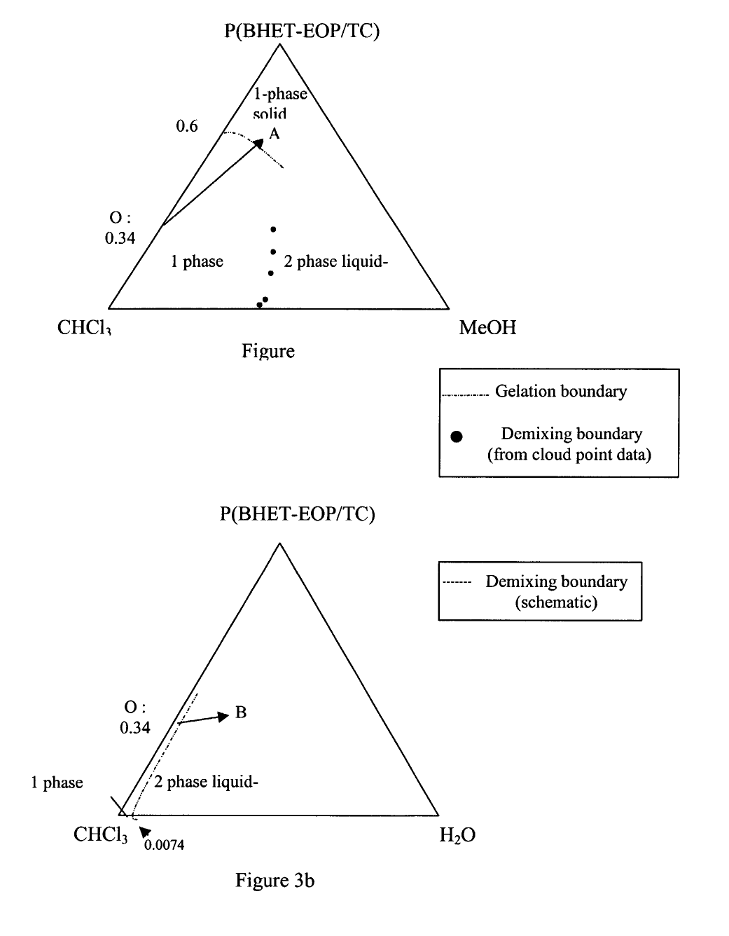

In order to achieve an improved method for microporosity control, one approach can be considered based on the ternary phase diagram, (FIG. 3). As discussed, the path that the system traverses within the liquid-liquid demixing region before it encounters the gelation-boundary would affect the resulting porosity. The length of the latter path may be adjustable by changing the coordinates of the demixing boundary. This can be achieved by using a mixture of non-solvents in the immersion bath. Methanol was chosen as the other non-solvent in addition to water as the two non-solvents are completely miscible in all proportions.

Observations made during immersion of the dip-coated mandrel into immersion baths containing different volume fractions of methanol and water indicate the phase changes that occur during the process. Upon immersion in precipitation baths containing less than 50% by volume of methanol, the coating became immediately white and opaque, indicating the process of phase separation. In contrast, for immersion in precipitation baths containing more than 50% by volume of methanol, the coating gelled to give a transparent gel. The rate of gelation was proportional to the concentration of methanol in the immersion bath. When the immersion bath contained equal volumes of methanol and water (50:50 MeOH/H₂O), the coating became opaque only gradually, over a period of 10 minutes. Part of this opacity was retained after the 10 minute period of air-drying. In contrast, coatings immersed in the other immersion baths became essentially transparent after air-drying.

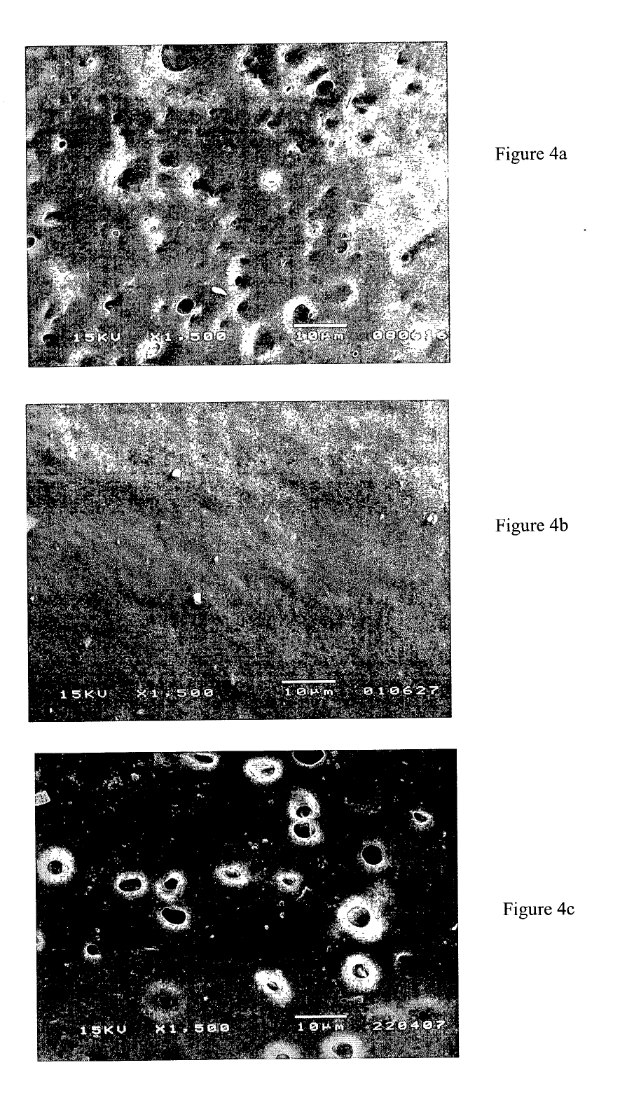

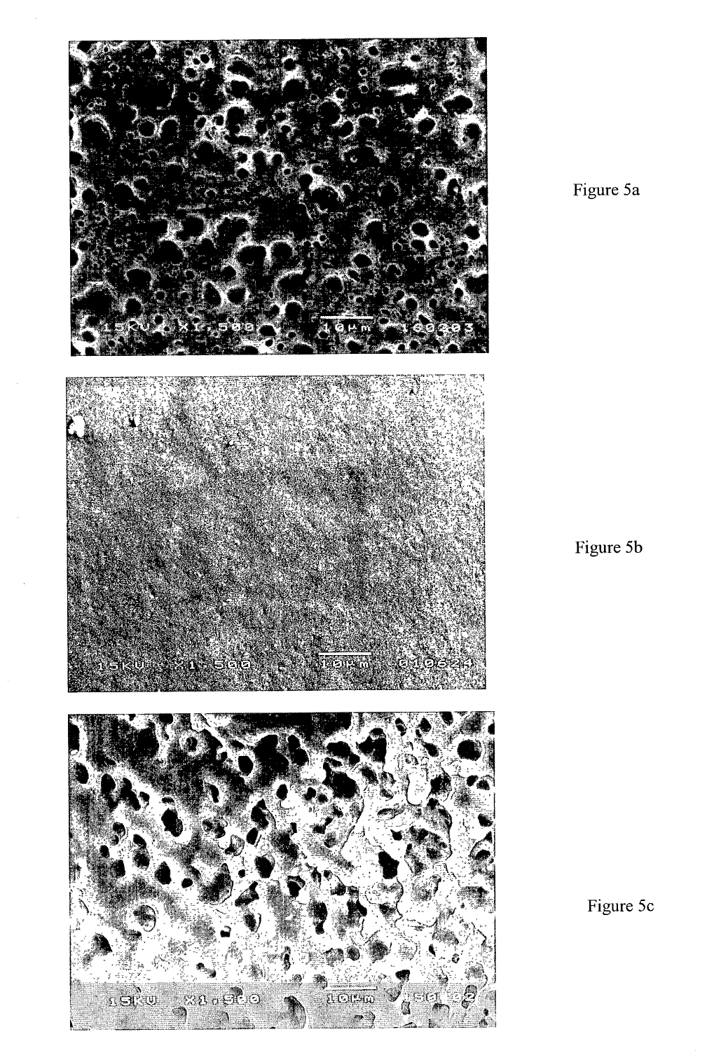

When >50% methanol volume fraction was employed in the immersion bath, gelation of the coating was observed, yielding a transparent gel. This can be explained by the relatively large quantity of methanol required to effect liquid-liquid demixing, methanol being a rather poor non-solvent for P(BHET-EOP/TC). This can be represented using the three-component, isothermal phase diagram illustrated in FIG. 3a. As determined by cloud point measurements, the boundary representing liquid-liquid demixing is some distance away from the polymer-solvent (P(BHET-EOP/TC)—CHCl₃) edge of the triangle. In other words, a relatively large percentage of methanol must be present in the system in order to effect the demixing phenomenon. This, coupled with the miscibility of methanol with chloroform which results in rapid efflux of solvent from the coating, brings the system into the gel region of the phase diagram. The path assumed by the system can be represented by the arrow OA. Once the gelation boundary is crossed, the polymer gels into a one-phase solid. Correspondingly, SEM analysis reveals a dense microstructure for the polymer surface. (FIG. 4a)

A totally different scenario presents when a low methanol volume fraction (<50%) is used as the immersion bath. Water, being only partially miscible with chloroform, penetrates the coating solution very slowly and only to a limited extent. Similarly, solvent efflux is slow and as a result, the process of gelation takes place very slowly. This ‘delay’ in gelation presents ample time for the process of liquid-liquid demixing to occur, as evidenced by the change in appearance of the coating from transparent to opaque upon immersion. As water is essentially a non-solvent for the polyphosphoester, just a small quantity of water added to the polymer solution (0.045% by wt) had resulted in turbidity. Hence, the demixing boundary for the PPE-CHCl₃-water system could not be determined. Nevertheless, it can be perceived that this boundary is located close to the polymer-solvent edge of the phase diagram, as shown schematically in FIG. 3b. In the 10-minute period of immersion employed in the procedure, the system goes only as far as the 2-phase liquid-liquid region where liquid-liquid demixing takes place but does not proceed to gelation. This is represented in the phase diagram by the path of the arrow OB.

Experimentally, air-drying of a coating immerse-precipitated in a pure water bath quickly led to a change in the coating from opaque back to transparent. As the phase-separated structure had not been frozen in place by gelation, removal of liquid from the polymer lean phase by evaporation rapidly led to a single phase material. Not surprisingly, gross observation revealed that coatings precipitated using <50% methanol volume fractions followed by vacuum drying exhibited dense microstructures. However, a coating precipitated in water and allowed to stand in water for several hours eventually gelled. A low degree of porosity is achievable by this method, as revealed by SEM of the polymer surface. (FIG. 4b)

A 50:50 MeOH/H₂O immersion bath enabled the system to achieve both liquid-liquid demixing and gelation within a period of 30 min. When the coating was immersed into the 50:50 MeOH/H₂O bath, it became immediately translucent upon immersion and proceeded to opaque after 5 minutes. The process of liquid-liquid demixing appeared to be slower compared with immersion in pure water, indicating that the demixing boundary had shifted away from the polymer-solvent edge of the ternary phase diagram. However, due to the presence of methanol, the system managed to gelate after 20 minutes in the immersion bath. Due to the ‘freezing’ of the 2-phase structure by the onset of gelation, the opacity was not completely removed by air-drying. The surface of the vacuum-dried samples revealed the presence of circular pores of a closed cell morphology (FIG. 4c).

A porous surface could be achieved by immersing the polymer coatings in water followed by freeze-drying (FIG. 5a). In contrast, when coatings were immerse precipitated in methanol and subsequently freeze-dried, a dense microstructure was obtained for the polymer surface (FIG. 5b). Evidently, substantial inclusion of water in the coating could not occur after it had gelated in methanol. When a 50:50 methanol-water mixture was used as the immersion bath, the freeze-dried specimen was porous and exhibited an almost reticulated, continuous morphology. (FIG. 5c) This seemingly better porosity may be because methanol, being mutually soluble in chloroform and water, improves the penetration of water (a good liquid-liquid demixer) into the coating.

The use of a combination of non-solvents to effect liquid-liquid demixing and gelation respectively may be highly useful for the generation of porous membranes by immersion of a supported polymer coating. Water, which is only sparingly soluble in chloroform, can be employed to effect liquid-liquid demixing. The second non-solvent, methanol, is used to accelerate the process of gelation that freezes the phase separated structure. This completes the sequence of physical changes that occurs during the formation of a porous membrane.

Biological Performance of PPE Conduits:

Biodegradable polymers with lower molecular weights can facilitate a fast degradation. One concern with using this kind of material to fabricate NGCs is the possibility of impeding nerve regeneration and/or impairing maturation of the regenerated nerves due to a lower mechanical stability and an early disintegration of a tube. PPE polymers used for NGCs of one embodiment have molecular weights ranging from 14,900 to 18,900, probably the lowest molecular weights for polymersused for NGC fabrication. Successful nerve regeneration has been observed within the conduits made from these polymers. When a fresh-cut nerve is repaired with such PPE conduits prefilled with PBS (Phosphate Buffer Solution), a solid structure that bridged the two nerve stumps formed within 3 days. This is much faster than that in a prior art PBS-prefilled silicone chamber, which takes 5 to 7 days before sufficient material accumulates to form a continuous bridge across a 10 mm gap [Williams et al., Neurochem Res. 1987, 851-860]. It is possible that the improved performance of the PPE conduits was caused by tube porosity and increased permeability due to tube cracks which allowed the influx of nutrients and growth factors from the surrounding environment and enhanced the constitution of the matrix.

Degradation of PPE Conduits:

PPE polymers degrade in the presence of nucleophiles, such as the hydroxyl ion. Their phosphoester bond is cleaved by hydrolysis at random sites along the polymer chains. Two types of hydrolysis, surface verse bulk, have been described in the art. Surface hydrolysis is characterized by a linear decrease in mass together with little initial decrease in molecular weight while typical bulk erosion shows a rapid decrease in molecular weight before any significant mass loss. The parallel reduction in conduit mass and in Mw (molecular weight) as observed in the present invention suggests that both surface and bulk hydrolysis occur in PPE NGCs.

Continuous decrease in conduit mass by leaching out of degradation products will reduce the strength and toughness of a tube, ultimately resulting in a loss of structural integrity of a conduit under the pressure of surrounding structures. If this occurs too early, before a tissue capsule is formed over the conduit and a matrix cable is strongly attached to both stumps, the failure of regeneration would be a most likely consequence. Complete breakdown of the conduit is, however, desirable after the nerve has satisfactorily regenerated, to obviate the painful complications arising from nerve compression by a rigid conduit. As is disclosed herein, a NGC made from P(BHET-EOP/TC) is degradable. The difference in degradation rate between Type I and II conduits in the in vitro study described below can be explained based on the polydispersity indexes or the molecular weight distribution of polymers used. The polymer for fabrication of Type I conduits possessed a higher low molecular weight fraction compared to the one for Type II NGCs. This fact, in addition to its higher porosity, results in a higher degree of swelling for the Type I conduits. As such, the hydrolyzable linkages in the polymer become more accessible to be attacked by water molecules, leading to a higher degradation rate. A slower degradation rate of PPE NGCs in the body, in comparison with that in vitro, is probably due to the gradual formation of a fibrous tissue capsule around the implant. This may result in decreased accessibility of the polymer's ester linkages to water molecules and hence, a lower observed rate of degradation. Nevertheless, it was observed, in view of changes in molecular weights and SEM pictures, that degradation of the NGC implants did occur in the body.

The degradation rate of a P(BHET-EOP/TC) polymer is related to the phosphate content in its backbone. The higher the phosphate content, the faster the degradation rate of the polymer [Mao et al., Evaluation of polyphosphates and polyphosphonates as degradable biomaterials. J Biomed Mater Res, 1991;25:1151-67]. This feature has provided a simple way to control the time frame of degradation of a P(BHET-EOP/TC) conduit. Increase in the BHET-EOP part during polymer synthesis may provide more phosphate content, therefore speeding up degradation. It is also possible to reduce this part to slow down degradation of the polymer when persistence of structural integrity is required. The time required for structural disintegration of a biodegradable NGC is decided by the time needed for nerve regeneration, which is variable with defect length, nerve location, and patient age [12]. P(BHEP-EOP/TC) copolymers with different ratios could be a choice to meet different requirements in obtaining optimal degradation rates of NGCs.

Tube Swelling:

Tube swelling is often observed with biodegradable nerve guide conduits. In those tubes with a porous wall, the initial swelling may result from water uptake into the pores, which explains the relatively higher swelling experienced by the Type I NGCs disclosed herein. As tube degradation starts, lower molecular weight degradation products absorb water and enhance swelling. This may cause problems when the swelling is sufficient to distort the tube lumen. Such a deformation resulted in nerve compression and impeded outgrowth of regenerating nerves in nerve guides constructed of an amorphous copolymer of lactic acid-caprolactone [9]. A correlation between biodegradability and distortion was observed with biodegradable polyester tubes, showing that the more biodegradable a tube, the more likely it was to incur distortion and regeneration failure [25]. Ways to relieve the problem include increasing the internal diameter and reducing the wall thickness of the tube. These may, however, result in growth of fibrous tissue into the lumen or affect the mechanical strength of the tube.

A nerve guide that is less affected by swelling may have increased practical flexibility in clinical application. According to one embodiment of the invention an amorphous polymer P(BHET-EPO/TC) is used to fabricate guides. Neither tubes with a wall thickness of 170 μm nor those with a wall thickness of 240 μm showed any significant swelling. These data are consistent with those obtained from NGCs made from DegraPol, a biodegradable polyester urethane with a low degree of swelling [4]. Thus, the material used can influence the extent of swelling of a biodegradable synthetic tube. In the case of the P(BHET-EOP/TC) series of PPE polymers, changing the relative hydrophobicity by substituting ethylene glycol with other diols may affect swelling [15].

Crystallinity:

Crystallinity has been given attention among the various properties of degradable polymers suitable for NGC fabrication [4,11]. Mixing a crystallizable polymer with an amorphous one may improve the mechanical properties of a tube that allow easy suturing during implantation [4]. Since crystallinity decreases the solubility of polymers, this mixing may slow down tube degradation, which may in turn aggravate foreign-body reaction. Some copolymers obtained from L-lactide and 6-caprolactone show a gradual crystallization at room temperature, resulting in progressive hardening with time [26]. In the P(BHET-EOP/TC) series of PPE polymers, according to one embodiment of the present invention, those with a BHET-EOP/TC charging ratio higher than 50/50 are amorphous with no melting point. Only when the ratio is reduced to 50/50, a crystalline phase begins to form in the polymer (Tm=201° C.) [15]. Consistent with this, PPE polymers in which the BHET-EOP/TC ratio is 80/20 showed no crystallinity. This property allows for complete biodegradation of a P(BHET-EOP/TC) conduit.

Biocompatibility of PPE Conduits:

Low inflammatory responses have been reported for PPE polymers, characterized with minor encapsulation and slight or no lymphocyte, giant cell, or macrophage activity [13,15]. The cytotoxicity test of P(BHET-EOP/TC) showed no effects on morphology and proliferation of human embryonic kidney cells and no mutagenicity in the Ames test [15]. Studies were carried out in relation to embodiments of the present invention. These studies concentrated on the nerve system with several in vitro assessments of cytotoxicity and showed no toxic effects of P(BHET-EOP/TC) polymers and conduits on primary neurons and neuronal cell lines tested. The studies also included a sensitive immunostaining method using antibodies against the 68 kD neurofilament protein for detecting neuronal degeneration. No differences between experimental and control cultures in the NF68 staining is shown in this study, indicating that there are no adverse effects of P(BHET-EOP/TC) breakdown products on neurons.

The potential toxicity of a biodegradable NGC is a factor that should be considered together with in situ degradation rates and local tissue clearances. The examination of in situ tissue response to implanted P(BHET-EOP/TC) conduits has shown a low inflammatory reaction, comparable to previous reports. This low reaction is clinically desirable in minimizing adhesions of an implanted conduit to surrounding tissues, especially to tendons of an injured hand.

Improved Nerve Regeneration by Incorporation of Gene Delivery Systems:

Targeting CNS neurons by endocytosis at nerve termini and retrograde axonal transport following peripheral injection of therapeutic genes is non-invasive to the CNS regions. Many viruses, metals, proteins and large particulate agents, including polymer particles like dextran and latex, are capable of reaching CNS neuronal cell bodies through this route. Polyethylenimine (PEI)/DNA complexes have been loaded into nerve guide conduits and proximal nerve stumps were allowed to take up the complexes. Gene expression was detected in dorsal root ganglion neurons and spinal cord neurons. When genes whose products are trophic to neurite outgrowth were used, better regeneration of severed nerves was observed. DNA complexes may be prepared from some other cationic polymers, for example, poly-L-lysine or chitosan. Cationic lipid may also be used for DNA complex preparation.

Improved Nerve Regeneration by Incorporation of Sustained Protein Delivery Systems:

Various active factors may aid in stimulating and enhancing nerve regeneration. Preferable nerve growth enhancers are growth factors, such as nerve growth factor (NGF), brain derived neuro-trophic factor (BDNF), and ciliary neurotrophic factor (CNTF). NGF is a potent simulator of sensory nerve outgrowth. NGF-treated substrates can provide directional guidance for the regrowth axons towards specific areas. Diffusion gradients of NGF can promote directed neurite extension in two-dimensional cell culture. NGF is thought to mediate chemotaxis required for successful reestablishment of functional synaptic connections.

To achieve a prolonged release of NGF, it is encapsulated in microspheres composed of a PPE polymers. Microspheres may be fabricated from any other biocompatible polymers, for example, poly(lactic-co-glycolic acid). The diameters of microspheres of this embodiment of the invention range from 5 to 20 micrometers. These small diameters are necessary in suspending the microspheres within regenerated tissue cables, therefore providing a relatively even distribution of released NGF. Controlled release of bioactive NGF from these microspheres lasted for at least 3 months. These microspheres were loaded into the nerve guide conduits to allow a sustained action of NGF on regenerating nerves. In a preferred embodiment, at least 100 microgram of NGF is loaded into a 10 mm conduit.

### 6. EXAMPLES

### Example 1

### Conduit Fabrication

I.1: Dip-Coating/Immersion Precipitation Method

To obtain a porous type guide (Type I, surface porosity 35%), A 34% (w/w) solution of P(BHET-EOP/TC) (EOP/TC=80:20) in chloroform was prepared by magnetic stirring. A Teflon mandrel of diameter 1.5 mm was vertically dipped into the polymer solution by a mechanical linear head at a speed of 8.3 mm/s and allowed to remain in the solution for 30 s. The mandrel was withdrawn at 24 mm/s and immediately immersed into the non-solvent bath where it was allowed to stand for 10 min. The mandrel was subsequently rotated horizontally for 10 minutes to reduce variations in the wall thickness along the axis of the tube and at the same time, to facilitate the process of air-drying. The coated mandrels were equilibrated in water overnight, frozen at −20° C. and subsequently freeze-dried using a Modulyo Freeze-drying Unit at a pressure of 0.1 Torr for at least one week. Finally, the polymer coatings were removed from the mandrel and characterized, or used for the implantation experiments.

To obtain a dense type guide (Type II, surface porosity=8%), a similar procedure as described above was applied, with the exception that the prepared sample was not frozen before being subjected to the process of vacuum drying.

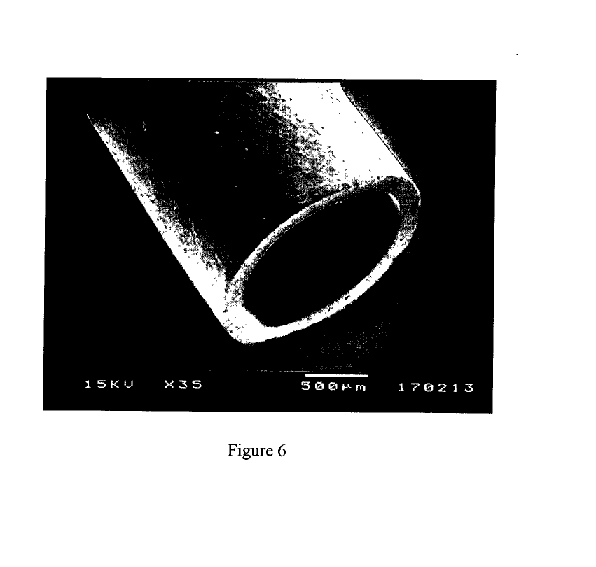

A typical P(BHET-EOP/TC) nerve conduit is shown in FIG. 6, possessing a diameter of 1.5 mm and wall thickness of approximately 100 μm. In the cross section of a nerve conduit (3 dip-coating steps) at higher magnification, the porous, layered structure of the conduit wall is apparent, each layer measuring approximately 25 μm. Another structural feature, i.e. finger-like cavities is observed as well at different regions of the conduit cross-section. These cavities are likely to improve the rate of mass transfer of permeant molecules through the conduit wall. The nerve conduit is asymmetric in terms of porosity. Its outer surface is clearly more microporous as compared to the luminal surface, where only scattered pores are apparent. An explanation for this may lie with the method of fabrication. Throughout the process of immersion precipitation, the luminal side is always in contact with the PTFE mandrel, whose surface interacts poorly with the aqueous phase of the demixed solution. The low porosity of the luminal surface thus comes as no surprise, knowing that it is sublimed ice that gives rise to the free volume during freeze-drying.

I.2: Film Rolling Method

A 20% (w/w) solution of P(BHET-EOP/TC) (EOP/TC=80:20) in chloroform was prepared by magnetic stirring. Ten ml. of this solution was cast into a mold with a base measurement of 10×7.5 cm, and the solvent was allowed to evaporate off in a fume hood for a period of 3 days. The polymer film could then be peeled off the mold and cut into strips measuring 2×1.6 cm. These strips were rolled around mandrels of the desired NGC dimensions and sealed by means of chloroform or dimethyl-formamide. The sealed tubes were vacuum-dried for at least 24 hours before use.

### Example II

### Control of Porosity

The approach used in this invention to control porosity of NGCs was based on the ternary phase diagram (FIG. 3). To determine demixing boundary and gelation point for ternary diagram, cloud point data for the determination of the demixing boundary was obtained by titration. Pure non-solvent was slowly added to different concentrations of the homogenous polymer solution at a constant temperature of 25° C. until permanent turbidity was detected visually. Care was taken to minimize the evaporation of CHCl₃ during the procedure. The gelation point for a P(BHET-EOP/TC) chloroform solution was determined by casting a small volume of the solution in an evaporating dish, measuring weight loss of the solution during chloroform evaporation and plotting solution weight versus time. At the point of gelation, a drastic decrease in the rate of evaporation (represented by weight loss) occurs due to a slower rate of solvent mass transfer to the surface of the film.

The porosity of the polymer surfaces obtained via the various procedures was estimated by generating a random array of 20 coordinates using a microprocessor, upon which the respective SEM micrographs (at 1500× magnification) were superimposed. The surface porosity, expressed as percentage of surface area covered by pores was calculated by employing the statistically derived formula:

surface porosity (%) number of coordinates within pore region/total number of coordinates×100%

The procedure was performed three times for each micrograph to obtain the mean surface porosity.

It was found that coatings immersed in water and subjected to freeze-drying were porous, while those that were vacuum dried were dense. Coatings immersed in pure methanol were dense no matter whether they were freeze or vacuum dried. Coatings immersed in MeOH-water mixtures exhibited varying degrees of porosity, depending on the MeOH-water ratio employed.

To obtain porosity with gradient precipitation, the dip-coated mandrel was first immersed in 15 ml baths of different initial methanol concentrations, ranging between 0 to 60% (v/v) methanol. For each system, the percentage of methanol in the bath was increased gradually to 70% by incremental addition of 5 ml methanol at 15 minute intervals. In this way, different degrees of liquid-liquid demixing could be achieved (proportional to concentration of water in the bath), while it could also be ensured that all the systems proceeded to gelation.

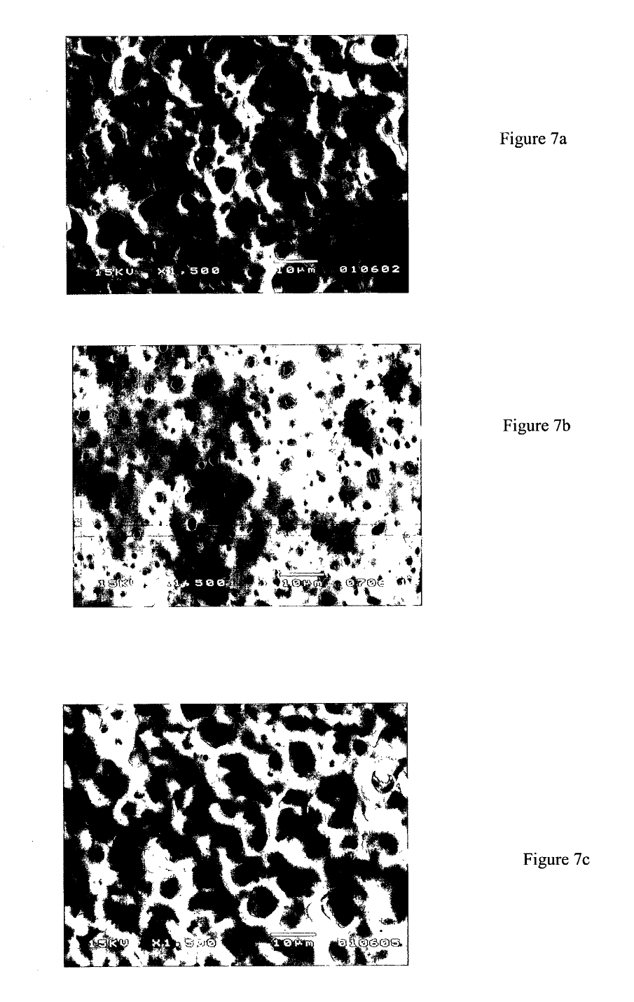

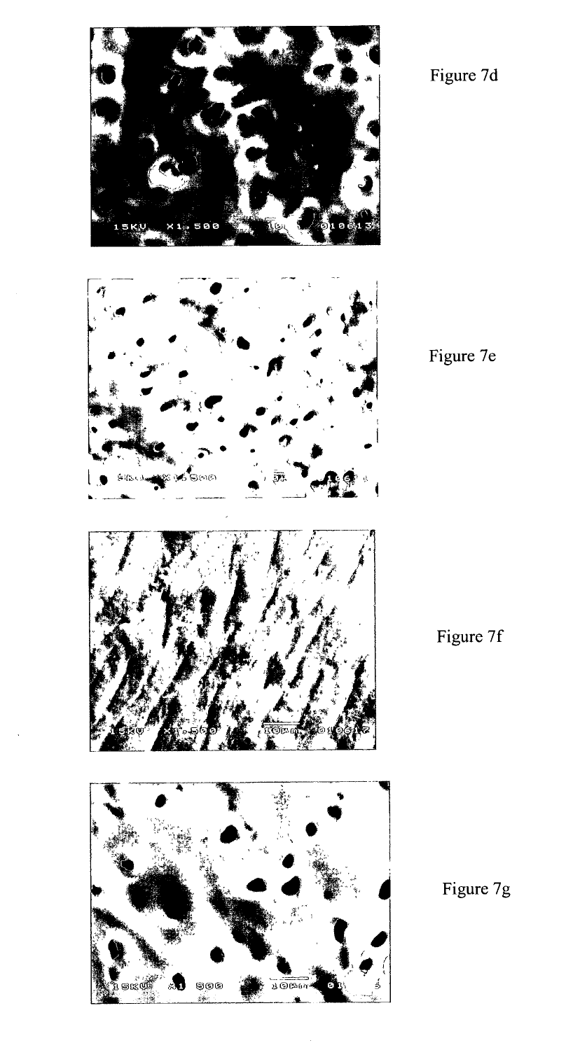

The surface porosity of a P(BHET-EOP/TC) coating can be controlled within the range of approximately 2 to 58% by employing the ‘gradient precipitation’ procedure described above. The corresponding SEM micrographs are shown in FIGS. 7a-g. When 0% methanol (pure water) was used as the initial immersion bath, the surface of the polymer coatings exhibited a highly porous morphology with a rugged surface and interconnected pores. As the concentration of methanol in the initial bath was increased, the surface porosity exhibited a corresponding decrease.

### Example III

### Permeability of Conduit

Permeability experiments were performed in order to relate the observed porosities of the conduits to their respective permeabilities, in terms of molecular weight cut-off and permeation constant. Two types of conduits were used, Batch 1 and Batch 2, fabricated from Type 1 and Type 2 polymers respectively. The surface porosity of the Batch 1 conduit was measured to be 35% while that of the Batch 2 conduit was measured to be 8% (see FIG. 2b,c)

A permeability experiment was carried out as follows.

One end of the nerve conduit was sealed by means of a water-proof sealant (Selleys All Clear® copolymer sealant). After the sealant had been allowed to dry, 10 μl of FITC-dextran (M_{w} 4,400) solution in pH 7.4 phosphate buffered saline was introduced into the lumen of the nerve conduit by means of a syringe needle. The other end of the conduit was then similarly sealed and allowed to dry. The sealed tube containing the FITC-dextran solution was then immersed in 1.5 ml of pH 7.4 phosphate buffered saline contained in a 2 ml Eppendorf vial. At fixed intervals of time, 0.1 ml of supernatant was transferred to a microplate reader (SLT Rainbow) and the absorbance read at 492 nm. The procedure was repeated using FITC dextran of a higher molecular weight (M_{w} 12 000). The permeability constant of the conduit wall was estimated by employing the formula:

J/A=(P/X)Δc

where

J=mass flux per unit time

A=area of the membrane

P=permeability constant

X=thickness of the membrane

Δc=concentration difference across the membrane

The concentration vs time curve was fitted to a hyperbolic function of the form y=ax/(bx+c). The value of J was subsequently obtained by calculating the gradient of the concentration-time curve at the origin, assuming a negligible lag time for the establishment of an equilibrium at the membrane-solution interface.

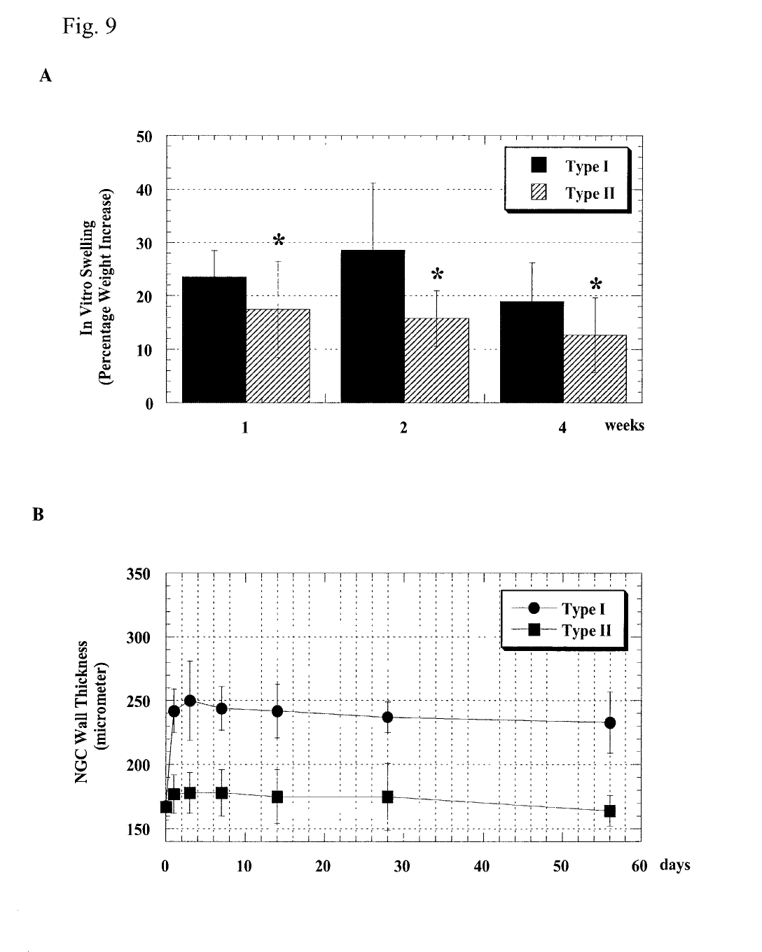

The results demonstrated that the Batch 2 conduit was impermeable to FITC-dextran of molecular weight 4 400. On the other hand, the Batch 1 conduit was permeable to FITC-dextran of M_{w} 4 400 but exhibited cut-off for FITC-dextran of M_{w} 12 000. The release profile for FITC-dextran by the Batch 1 conduit is shown in FIG. 9. From the graph, the permeation constant (J) for FITC-dextran of M_{w} 4 400 was estimated at 2.8×10⁻⁹ cm² s⁻¹. In the last section, reference was made to the asymmetric nature of the conduit wall. It is believed that the luminal layer acts as a semi-permeable membrane that mainly confers molecular mass selectivity to the conduit wall, whereas the porous outer wall region controls the rate of permeation and acts as a backing support. This contrasts with the typical asymmetric membrane where the perm-selective layer is usually the outer skin layer. The permeability experiments show that conduits of different porosities obtained by immersion precipitation demonstrate differences at a functional level.

### Example IV

### Biocompatibility of Conduits

In vitro cytotoxicity and tissue histological analyses were used for biocompatibility assessment of the P(BHET-EOP/TC) and the NGCs made with this polymer. Cell types used in this study included rat primary neurons from the dorsal root ganglion (DRG), the hippocampus and the cerebral cortex, undifferentiated rat pheochromocytoma PC12 cells and neuronally differentiated PC12 cells, as well as rat primary fibroblast cells. The cells were cultured in DMEM supplement with 10% fetal calf serum. For PC12 cells, 5% of horse serum was also added. Extracts for in vitro assessment of neuronal cytotoxicity were prepared by placing 5, 10 or 20 mg of P(BHET-EOP/TC) or conduits in 200 μl of the cell culture medium for 3 days with an extraction temperature of 37° C. The materials were sterilized with 70% ethanol before the extraction. Control solution was prepared in the same way without the test materials. To challenge cells with the extracts, the cells were grown to 50 to 80% confluency in a 24-well plate. The 200 μl of extracts plus 200 μl of fresh culture medium was added into each well of the plate. The cells were allowed to grow for another 3 days before morphological examination. At the end of experiments, some of the dishes with neuronal cells were rinsed with PBS and fixed with 4% paraformaldehyde and prepared for immunocytochemical analysis with a mouse monoclonal antibody against the 68 kD neurofilament protein. In vivo tissue response was evaluated after Toluidine blue staining of 1 μm semi-thin sections from conduit explants.

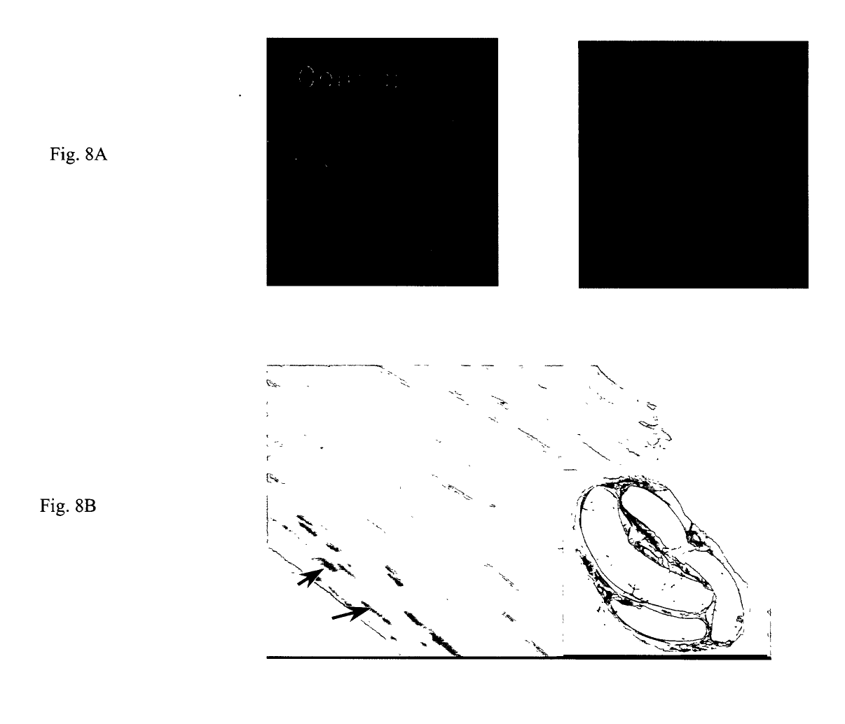

Compared to controls, extracts made from various amounts of P(BHET-EOP/TC) polymer and conduits induced neither morphological alteration nor cell death after treatment for 3 days in any of the cell types tested, including rat primary neurons from the dorsal root ganglion (DRG), the cerebral cortex and the hippocampus, undifferentiated rat pheochromocytoma PC12 cells and neuronally differentiated PC12 cells, as well as rat primary fibroblast cells. Immunostaining of DRG neurons or neuronal PC12 cells with antibodies against the 68 kD neurofilament protein, one of the major structural components within neurons, showed no difference between extract-treated and negative control cells in the intensity and pattern of the staining. In one experiment, thirty mg of the polymer, which is about 4 times of a conduit weight, was extracted for 2 weeks in PBS at 37° C. and primary DRG neurons were treated with the extract for 7 days. No deleterious effects were observed after the immunostaining of the 68 kD neurofilament proteins (FIG. 8A).

The assessment of the tissue response to PPE-made conduits was conducted after histological staining of the explants collected at various time points post-implantation. Two types of tubes displayed a similar effect. The formation of a thin fibrous tissue capsule around the tube could be seen at 3 days. New capillaries had penetrated and dispersed within the tube structure within the first 4 weeks. An initial inflammatory response during this period was characterized with a weak accumulation of lymphocytes and macrophages. This was more pronounced in Type I conduits. At 3 months, the inflammatory reaction was not notable. A well-organized fibrous tissue capsule was present on the outer surface of the conduit, with about 8 layers of fibroblasts and a thickness of about 20 μm (FIG. 8B). Some macrophages could be seen between the fibrous layers and the conduits. None of the implanted conduits triggered edema or necrosis.

### Example V

### Degradation of Conduits

To measure swelling of NGCs, conduits were placed in phosphate buffer pH 7.4 at 37° C. At selected time points, the conduits were removed from the solution, blotted with an absorbent tissue and weighted for weight increase. Seven samples were used for Type I and 8 for Type II conduits. Due to the breakage of Type I conduits, weight increase for swelling was not measured after 4 weeks. The percentage of water absorption (WA%) was calculated according to the following equation:

WA(%)=100×(Ws−Wr)/Wr

where Wr=residual weight of the dry conduit and Ws=weight of swollen conduit. Swelling was also analyzed by measuring changes of wall thickness and tube diameter of conduits using UTHSCSA Image Tool.

To measure weight loss, conduits were placed in phosphate buffer pH 7.4 at 37° C. At selected time points, the solution was removed and the conduits were dried to constant weight under vacuum and weighted. The percentage of weight loss (WL%) was calculated according to the following equation:

WL(%)=100× (Wo−Wr)/Wo

where Wo=initial weight and Wr=residual weight of the dry conduit.

To measure changes in molecular weight, the conduits were rinsed in water, dried under vacuum, and dissolved in chloroform. NGC explants from the rat body were separated from surrounding tissue, treated with 0.5 mg/ml of collagenase Type II for 24 hours to remove fibrous tissue and other protein impurities and subsequently treated in the same way as the in vitro samples for measurement of molecular weight. Gel permeation chromatography (GPC) was performed in chloroform with respect to polystyrene standards to determine the weight-average molecular weight (Mw), number-average molecular weight (Mn) and polydispersity index (PI).

To examine the morphology of the NGC explants with scanning electron microscopy (SEM), the samples were gold-coated using Fine Coat Ion Sputter JFC-1100 and micrographs were obtained using a JEOL JSM-T220A scanning electron microscope at an accelerating voltage of 15 kV.

Samples for differential scanning calorimetry (DSC) were also enzymatically treated to remove protein impurities, as described above. DSC was preformed on a DSC 2010 Differential Scanning Calorimeter with a refrigerated cooling system (TA Instruments, New Castle DE, USA). Thermograms were obtained for the temperature from −20° C. to 200° C., at a heating rate of 10° C./min.

The data were subjected to an analysis of variance (ANOVA) using the SPSS statistical software. Multiple comparison tests were performed with Tukey tests where ANOVA showed overall statistical significance.

V. 1: In vitro Degradation

The phosphoester bond in a PPE backbone is cleaved by water. Thus the more readily water penetrates, the greater the bond cleavage and the faster its degradation rate. Yet deformation of a conduit caused by swelling could impede outgrowth of regenerating nerves. In vitro experiments were therefore conducted to characterize swelling of PPE conduits with different surface porosities. In one experiment, weight increases were measured after placing conduits in PBS at 37° C. for various periods of time. After 4 weeks, Type I conduits that have a surface porosity of 35% had a 30% weight increase while Type II, which porosity is 8%, swelled slightly more than 15%. In vitro swelling of those conduits was also characterized by measuring wall thickness. A forty-five percent increase of wall thickness was observed in Type I conduits while no significant increase was seen for Type II (FIG. 9). Tube diameter was not affected in both groups of conduits.

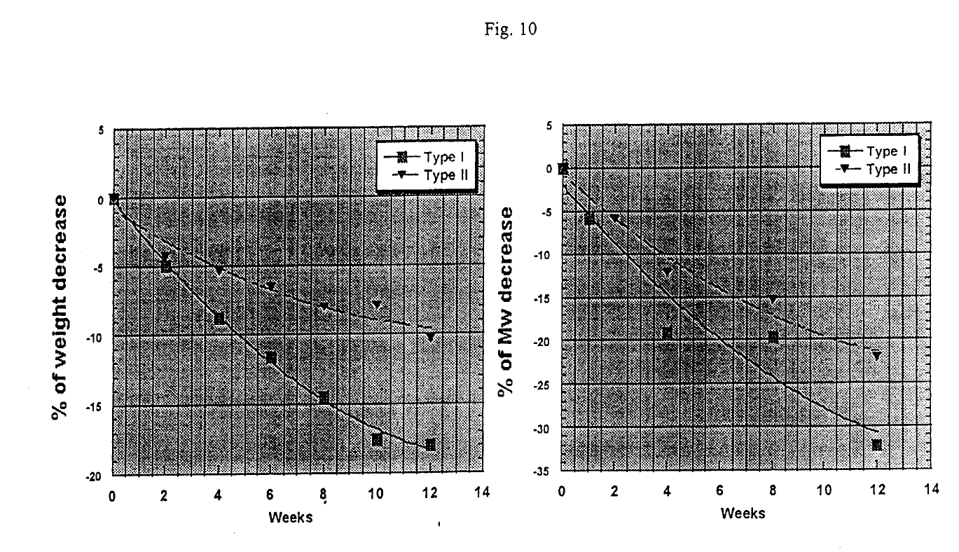

Degradation of the PPE conduits in PBS at 37° C. corresponded well to their swelling extent, i.e. when more water penetrated into a conduit, greater degradation occurred. Weight loss and decrease in weight-average molecular weight in a time-dependent manner documented this (FIG. 10). About 20% of weight loss was seen in Type I conduits after 12 weeks in PBS. Corresponding loss in Type II was 10%. GPC analysis showed that the higher molecular weight fraction of P(BHET-EOP/TC) had shifted to the lower end, confirming cleavage of polymer chains. For Type I NGC, the weight-average molecular weight had decreased from 15,000 to 10,000 after 12 weeks, a drop of 33%. For Type I NGC, the molecular weight dropped 24%, from 17,000 to 13,000 after 12 weeks.

V.2: In vivo Degradation of Conduits

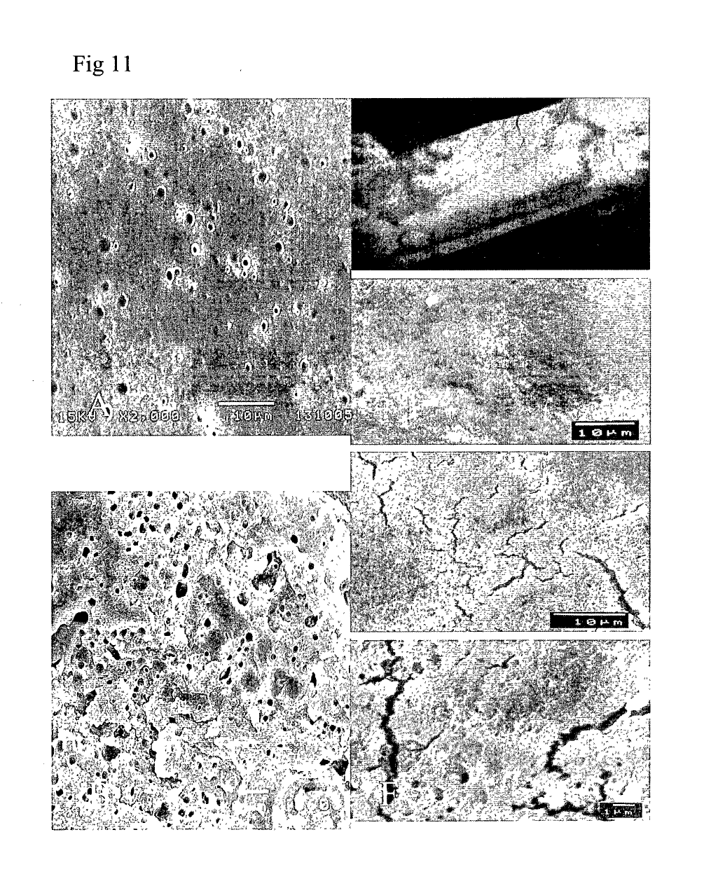

Type I conduits with surface porosity of 35% and Type II conduits of 8% porosity were used. Both types of tubes were rigid enough to be easily manipulated during microsurgery. After 1 month post-implantation, all tubes had fractured into pieces, which were held together by connective tissue. Under scanning electron microscope, some areas on the surface of Type I NGCs had changed from relatively smooth to very rough after 6 weeks already. This became more widely distributed after 3 months. The main microstructure change observed in Type II conduits at 3 months was numerous small cracks spreading all over the tube. The tube surface had become rough with an increased porosity (FIG. 11).

GPC analysis of NGC explants collected after 3 months of implantation showed a drop of 16% and 10% in the weight-average molecular weight in Type I (n=2) and II(n=3) conduits, respectively. The polydispersity index decreased from 2.22 to 1.76 for Type I and from 1.70 to 1.62 for Type II conduits.

DSC revealed the absence of any crystalline melting peaks for those material, which would have been expected in the range (−20° C. to 200° C.) analyzed if crystallinity had been present in the samples. This implies that crystallinity did not develop in a P(BHET-EOP/TC)NGC in vivo, at least up to a time period of 3 months.

### Example VI

### Nerve Regeneration Within Conduits

Twenty-six of male Wistar rats (200 to 250 g) were used for conduit implantation, 8 for Type 1 and 18 for Type II conduits. After anaesthetizing with pentobarbital, the right sciatic nerve of the rat was exposed through a 3 cm long skin incision on the thigh and retraction of the gluteus maximus muscle. The nerve was freed from surrounding tissue and transected at the midthigh level, proximal to the tibial and peroneal bifurcation. A 5 to 7 mm piece of the nerve was removed and then the proximal and distal nerve stumps were pulled 2 mm into each opening of the PPE tubes, leaving a 10-mm interstump gap. The two stumps were fixed within the tubes with a single 10-0 perineurial suture (Ethilon). Before the proximal stump was pulled into the tube opening, the tube was filled with saline. The surgery was performed under an Olympus operating microscope. The muscle layers were closed with 4/0 silk sutures and the skin closed with Michel clips.

One or three months after implantation, the sciatic nerves together with tubes were reexposed and carefully isolated from the surrounding tissue. The nerve segment distal to the tube was pinched in a pair of forceps. Contraction of muscle on the back or retraction of the leg indicates the presence of regenerating sensory fibers in the pinched segment. The rats were then sacrificed with an overdose of pentobarbital. The PPE conduits were excised together with nerves. After removing the conduits, the regenerated nerves were fixed with 4% paraformaldehyde and 2.5% glutaraldehyde in PBS for overnight before immunostaining. Twenty μm-thick longitudinal sections were cut on a cryostat and collected onto gelatin-coated glass slides. The sections were stained with mouse monoclonal antibodies against the 68 kD neurofilament protein. For morphometric analyses, the nerves were fixed for at least 3 days in the above solution and then post-fixed with 1% osmium tetroxide. After dehydration and embedding in Epon, the nerves were cut into 1 μm thickness of transverse sections, and stained with toluidine blue. Quantitative evaluation was carried out at the middle of the regenerated nerve cables using UTHSCSA Image Tool (The University of Texas Health Science Center in San Antonio). For each sample, 6 areas of about 500 μm² from two cross sections were evaluated.

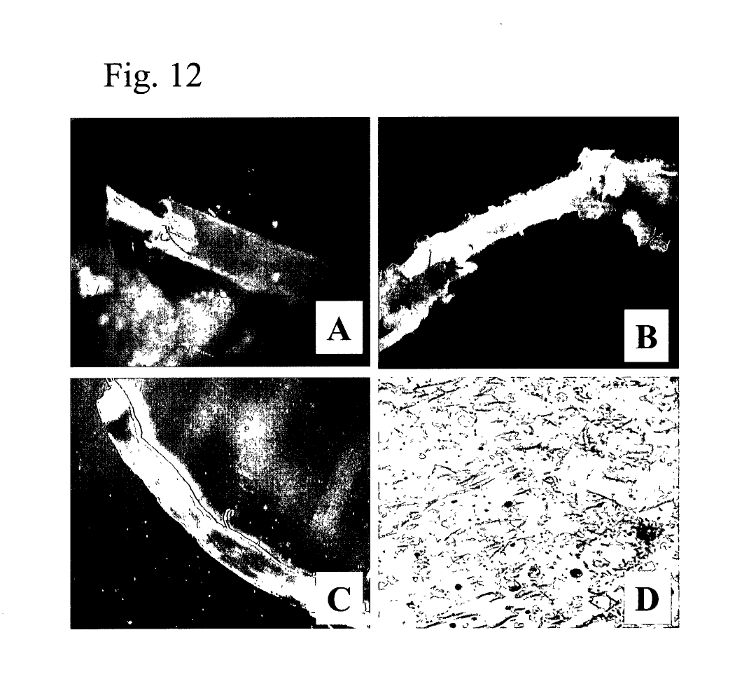

One day after implantation, some of the NGC chambers (3 out of 4 Type II conduits) had become filled with a solid structure that bridged the two nerve stumps. This structure appeared like blood clot and was loosely attached to the stumps. At day 3, the solid structure was present in all tubes examined (FIG. 12B). By microscopic examination, small threads distributed with a predominantly longitudinal orientation were observed (FIG. 12C), suggesting an accumulation of fibrin matrices [18,19]. The structure had become firmly connected with the stumps 5 days post-implantation. Regenerated axons could be observed 2 weeks later in the proximal part of regenerated cables and one month later in the distal sciatic stump, 5 mm distal to the suture line. Most of them were unmyelinated, present together with numerous Schwann cells (data not shown).

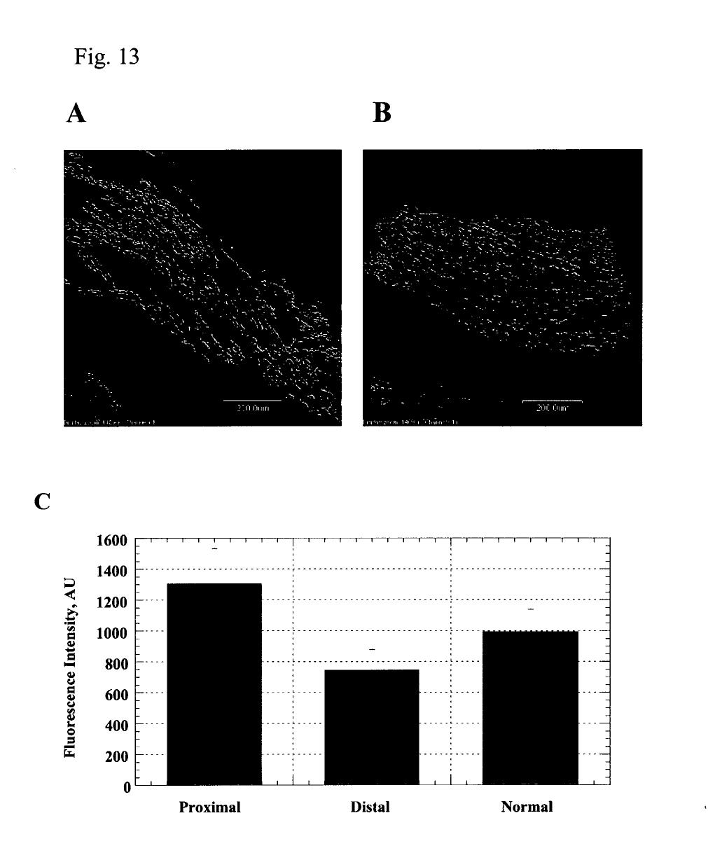

After three months, positive reflex responses were observed in 40% of the rats that were implanted with Type I and 92% of those with Type II conduits when the nerve trunks distal to the conduits were pinched in the anesthetized animals. All these rats had a regenerated cable inside the conduits, which had bridged a 10 mm gap between the nerve stumps (FIG. 12D). The regenerated cables were centrally located within the conduits, surrounded by a fine epineurium. The cables contained numerous fascicles of myelinated as well as unmyelinated axons. Most of the axons in the distal nerve trunks were already myelinated. Immunostaining with an antibody against the NF68 protein confirmed axon distribution through the whole regenerated cables three months after implantation. The staining was stronger than that in the normal undamaged control nerve at the proximal nerve trunks but weaker at the distal part (FIG. 13).

Transverse sections through the mid-point of the 10 mm gaps were analyzed to determine the number of myelinated axons one and three months after implantation (Table 1). The Type I conduits with a higher surface porosity had a higher fibre population than Type II conduit, which is very close to the value from the normal nerves by 3 months post-implantation.

TABLE 1
Morphemetric Analysis of the Regenerated Nerves at the
Midpoint of Conduits With Different Surface Porosity.

| PPE NGC | Porosity | Implantation Period | N | Fibre Population |
|---|---|---|---|---|
| Type I | 35% | 1 month | 4 | 3758 ± 1043 |
| Type II | 8% | 1 month | 7 | 2928 ± 1293 |
| Type I | 35% | 3 months | 4 | 8080 ± 240 |
| Type II | 8% | 3 months | 11 | 6684 ± 2155 |
| Normal Nerve | — | — | 7 | 7991 ± 257 |

### Example VIII

### Nerve Regeneration within Conduits Loaded with Polyethylenimine/DNA Complexes

Plasmid pcDNA3.1/lacZ, pcDNA/NGF, pcDNA/BDNF and pcDNA3/Bcl-2, under control of the cytomegalovirus immediate early region promoter and enhancer, were used. All plasmids were expanded in E. coli DH5α strain and prepared using Qiagen plasmid Maxi-prep kit according to the supplier's protocol (Qiagen, Ontario, Canada). The quantity and quality of the purified plasmid DNA was assessed by optical density at 260 and 280 nm and by electrophoresis in 1% agarose gel. The purified plasmid DNA was re-suspended in TE buffer and kept in aliquots at a concentration of 1 or 2 mg/ml.

Plasmid DNA was diluted in 5% glucose to the chosen concentrations. PEI (25 k, Sigma) was used as 0.1 M aqueous stock solution. Relative amounts of PEI to DNA were 10 equivalents of PEI nitrogen per DNA phosphate (N/P=10/1). Complexes were formed by adding the appropriate amount of PEI solution into DNA solution, mixing with brief vortex and waiting for 15 min at room temperature. For each loading, 20 μl of 5% glucose solution containing 4 μg of plasmid DNA complexed with PEI or other DNA carriers were used per conduit.

Wistar rats (200 to 250 g) were used for conduit implantation. After anaesthetizing with pentobarbital, the right sciatic nerve of the rat was exposed through a 3 cm long skin incision on the thigh and retraction of the gluteus maximus muscle. The nerve was freed from surrounding tissue and transected at the midthigh level, proximal to the tibial and peroneal bifurcation. A 5 to 7 mm piece of the nerve was removed and then the proximal and distal nerve stumps were pulled 2 mm into each opening of the PPE tubes, leaving a 10-mm interstump gap. The two stumps were fixed within the tubes with a single 10-0 perineurial suture (Ethilon). Before the proximal stump was pulled into the tube opening, the tube was filled with polymer/DNA complexes solution. The surgery was performed under an Olympus operating microscope. The muscle layers were closed with 4/0 silk sutures and the skin closed with Michel clips.

One or two days after conduit implantation, the rats were anesthetized with pentobarbital sodium, and perfused transcardially by saline solution (200 ml) followed by 150 ml of ice-cold fixative (4% paraformaldehyde, 0.05% glutaraldehyde in PBS). The dorsal root ganglion and spinal cord were removed and post-fixed in the same fixative for 1 day at 4° C., followed by 1 or 2 days cryoprotection in 30% sucrosein PBS until the tissues completely sank. Finally they were embedded in O.C.T. (tissue freezing medium, Miles Inc. USA). The coronal sections (20 to 30 μm) were cut at −20° C. in a cryostat. Sections were rinsed three times in PBS with 1 mM MgCl₂ (pH 7.8) and incubated for 8 hours at 37° C. in X-gal (5-bromo-4-chloro-3-indoyl-β-galactosidase) reaction mixture (Invitrogen, Carlsbad, Calif.) to detect lacZ gene expression.

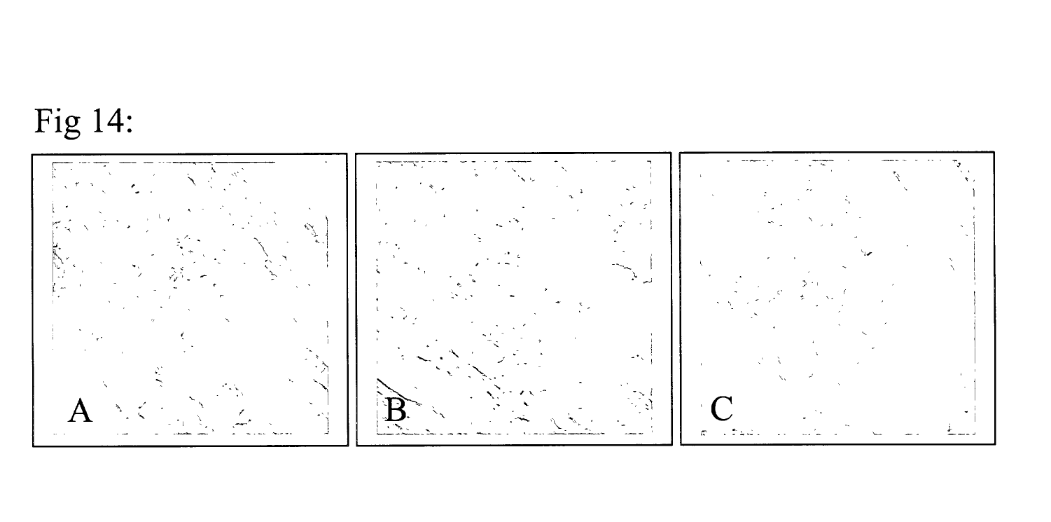

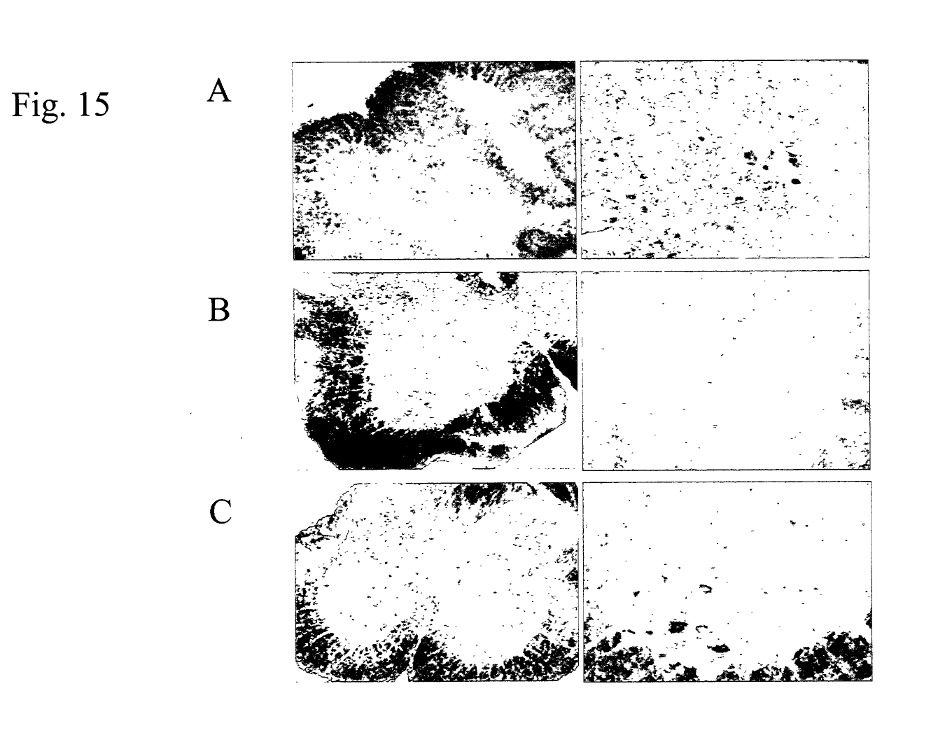

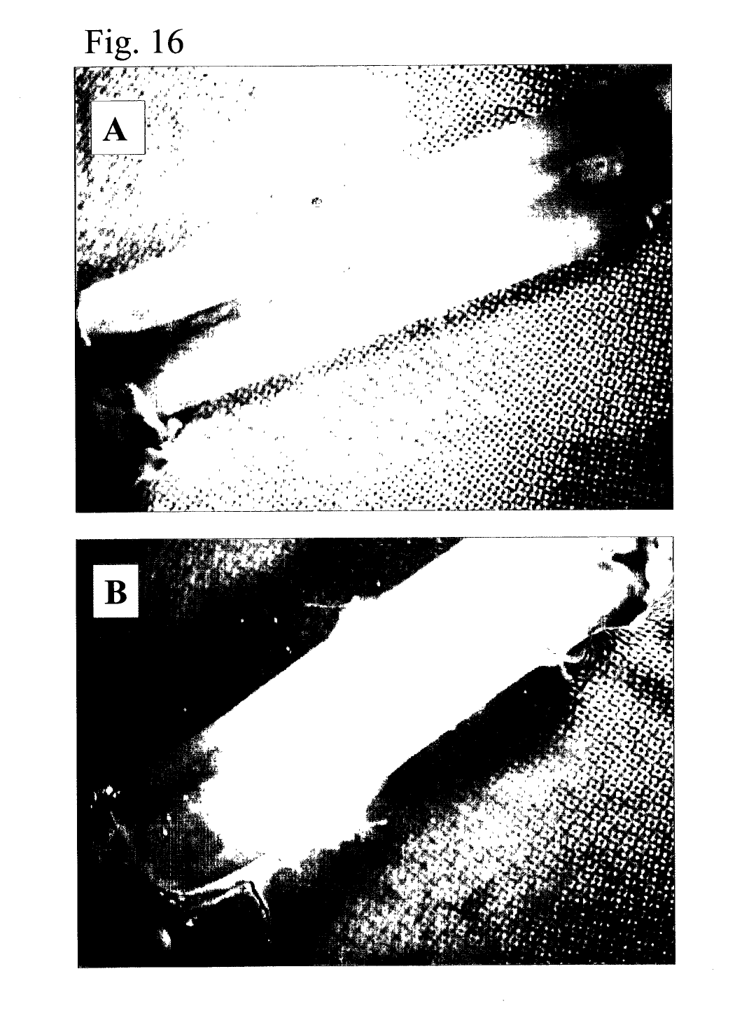

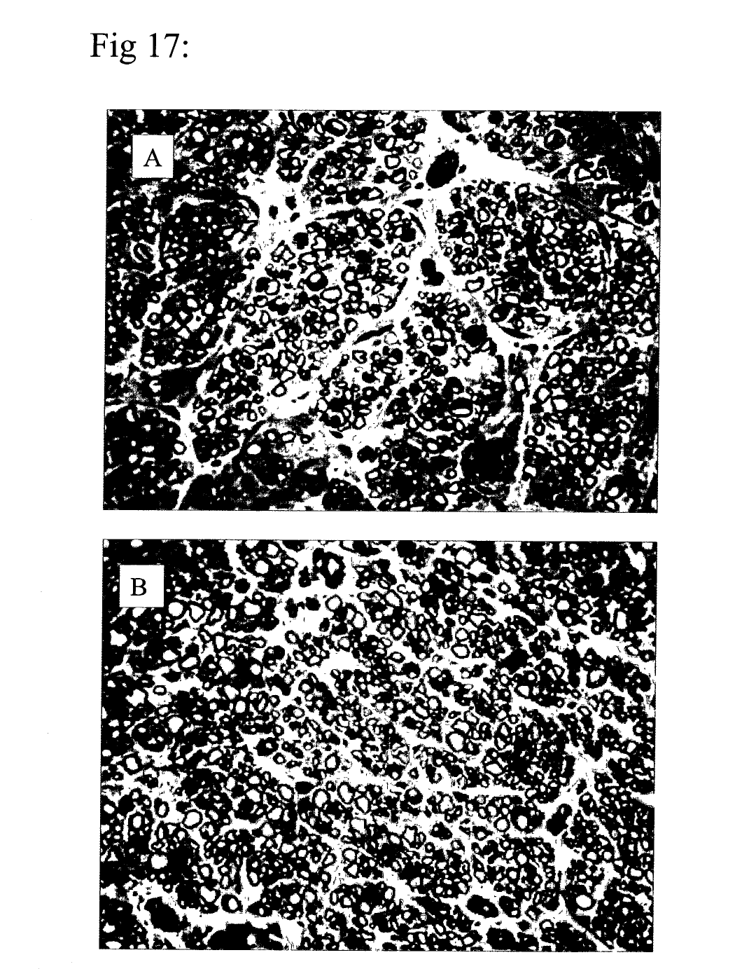

The lacZ gene expression was detectable in the dorsal root ganglion neurons one day after conduit implantation. A high level expression was found 24 hours after implantation, with about 80-90% of the dorsal root ganglion neurons positive in β-galactosidase (β-gal) staining (FIG. 14). The staining was also observed in the cytoplasm of large and small motor neurons in the spinal cord (FIG. 15). BDNF has been documented previously to be able to promote neurite outgrowth. The BDNF cDNA was complexed with polyethylenimine and loaded within the conduits. One month later, regenerated nerve cables within the conduits showed bigger diameters compared with those in conduits without the functional gene (FIG. 16). A higher density of fibre was noted in the conduits loaded with BDNF cDNA complexes (FIG. 17).

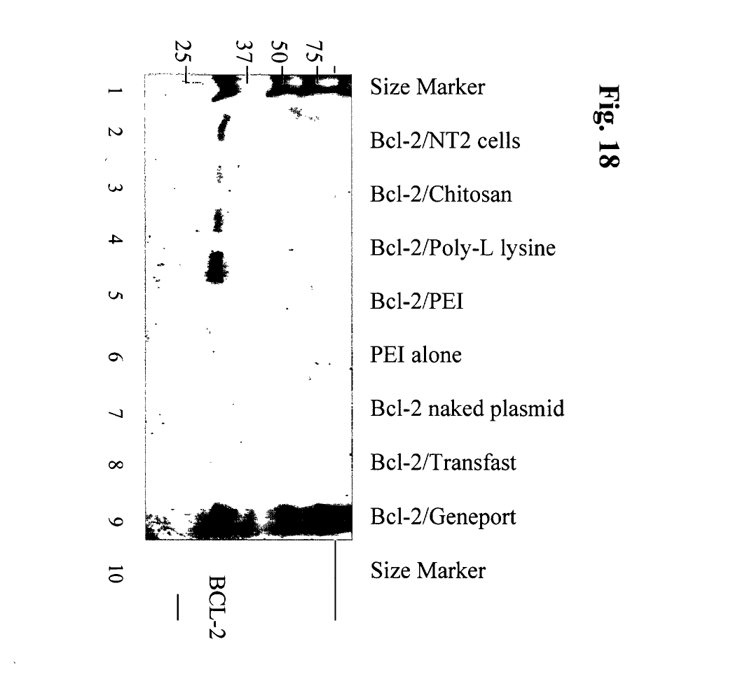

In addition to polyethylenimine, other cationic polymers or lipids may also be used to deliver genes through endocytosis and retrograde axonal transport. The gene expression of human Bcl-2 was used to compare their relative transfer efficiency. Optimal ratios between DNA and poly-L-lysine, chitosan, Transfast or GenePORTER were tested in agarose gel electrophoresis and COS7 cell transfection. The same amounts of pcDNA3/Bcl-2 were then complexed with those polymers and lipids under the conditions optimized for each individual agent and injected into the tongue. The proteins extracted from the brainstem 2 days after injection were subjected to western blotting (FIG. 18). The highest expression level was obtained with PEI, which is at least two times higher than that mediated with poly-L-lysine and chitosan as showed by densitomeric analysis. Very weak expression was observed with two cationic lipids, Transfast- and GenePORTER. Injection of free plasmid DNA into the tongue did not gain detectable Bcl-2 expression in the brainstem.

### Example VII

### Nerve Regeneration within Conduits Loaded with NGF Microspheres

Recombinant human nerve growth factor was purchased from Sigma. To make microspheres, 50 mg of PPE polymer was dissolved in 1 ml of methylene chloride. Two microgram of NGF together with 0.6 mg of BSA was dissolved in 30 μl of water. This mixture was added to the dissolved polymers during vortexing and the solution was sonicated for 20 seconds to give a homogeneous milky emulsion. Five ml of 1% to 10% PVA-5%NaCl was quickly added to this emulsion and vortexed for 30 seconds at high speed. This double-emulsion was poured into a 50-ml tube with 20 ml of 0.3% PVA-5%NaCl solution and mixed vigorously. The mixture was stirred for 3 hours to allow microsphere formation. The microspheres were collected by centrifuging the solution at 2000 rpm for 10 min. The microspheres were rapidly frozen in liquid nitrogen and then lyophilized for 24 hours.

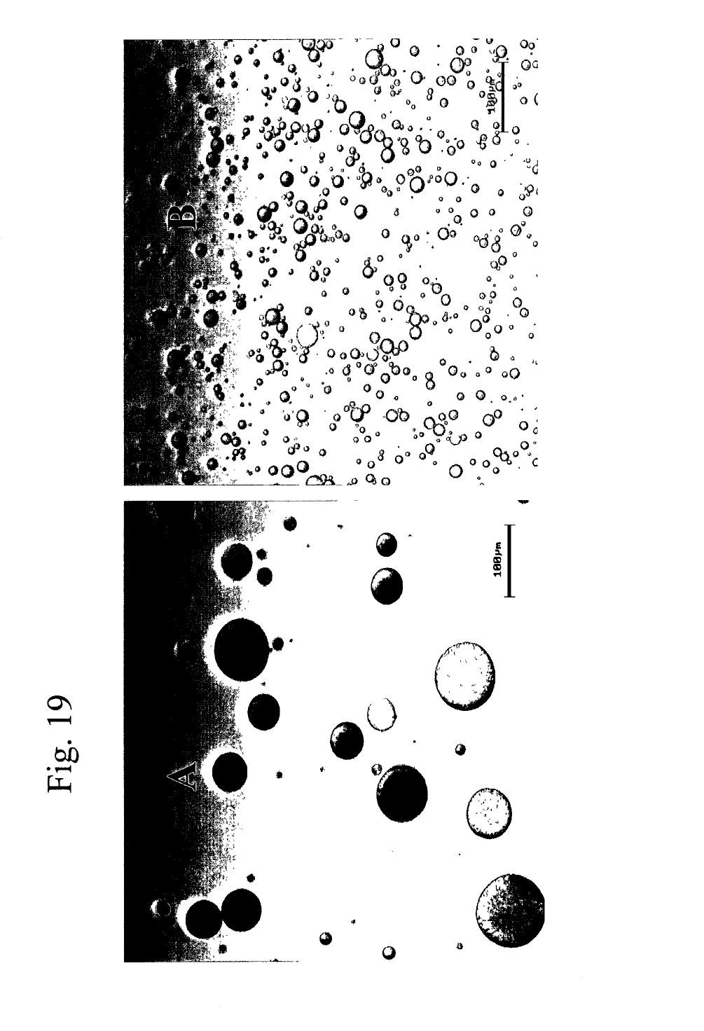

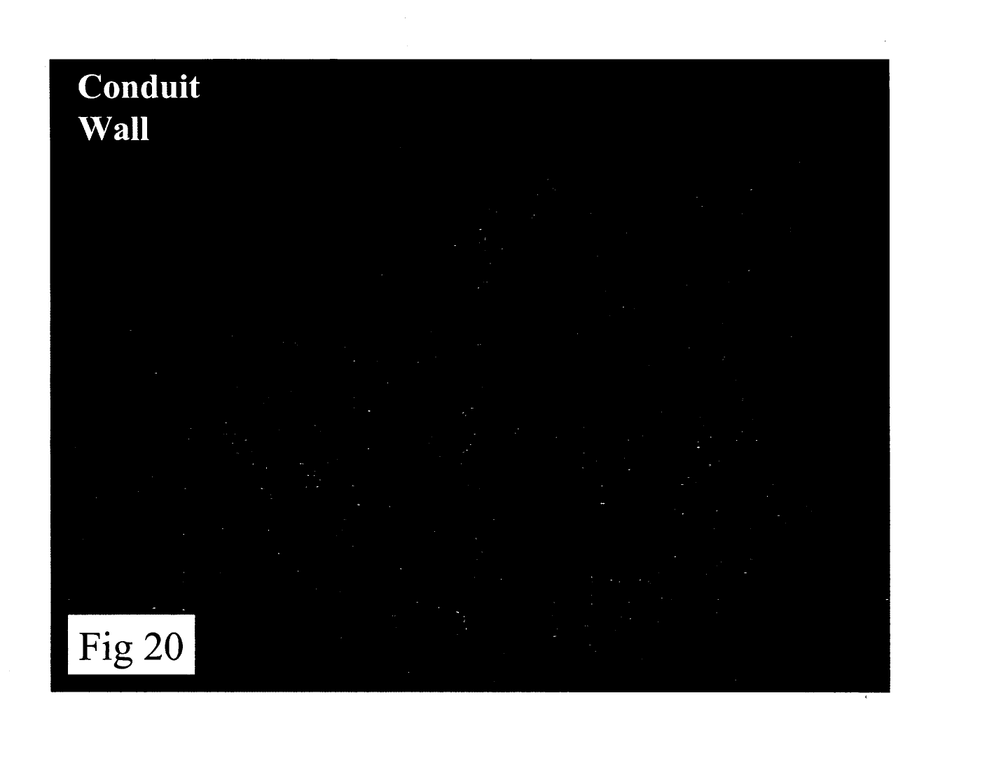

To enhance the suspension of microspheres in conduit solutions and to reduce the possible disturbing effects of big microspheres on neurite outgrowth, the microspheres used in the NGCs should have a smaller diameter. It was confirmed that using 10% (w/v) PVA as an emulsion stabilizer was necessary to prepare microspheres with diameters around 10 μm. (FIG. 19). It was further confirmed that, with such a small diameter, the microspheres may suspended within the regenerated tissue cables after loaded into NGCs (FIG. 20).

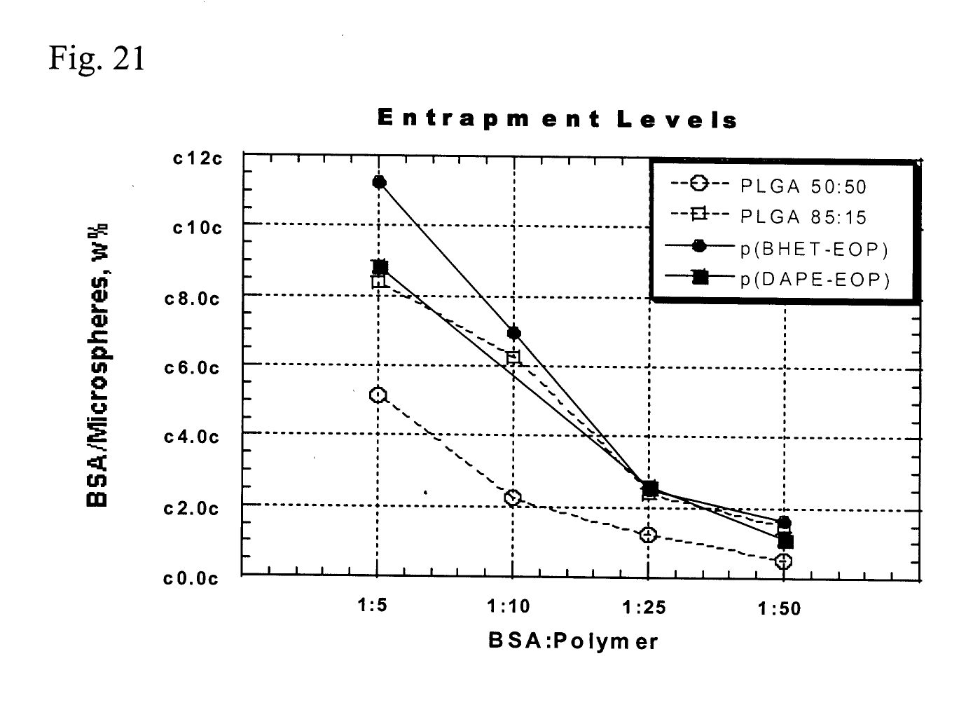

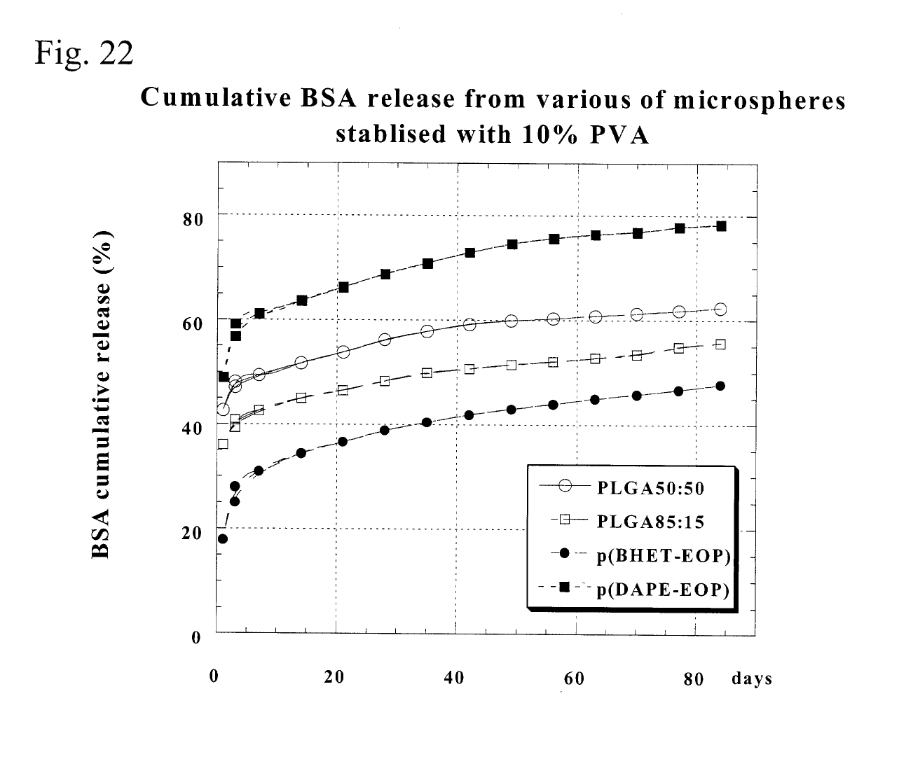

Four types of biodegradable polymers were used to prepare microspheres. The efficiency of encapsulation differed for each polymer was investigated, with two PPE polymers: p(BHET-EOP) and a comparative polymer, p(DAPE-EOP), having higher efficiency of protein encapsulation (FIG. 21). The greatest normalized cumulative release of proteins was observed from p(DAPE-EOP) (FIG. 22).

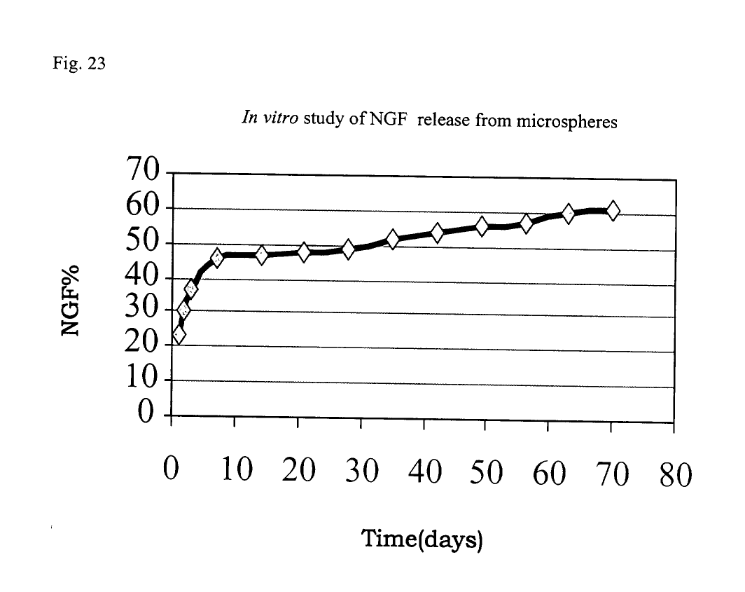

The rate of NGF release from the microspheres was determined by incubating them with PBS in a cell culture plate. The plate was placed at 37° C. Periodically, the buffer was replaced with fresh buffer. The concentration of NGF in the buffer was determined by NGF ELISA. Microspheres with an average diameter of 10 μM gave an immediate burst that about 50% of NGF was released within the first 24 hours. This immediate release was followed by a slow but sustained release of NGF for at least 3 months (FIG. 23). The released NGF was exposed to pheochromocytoma PC12 cells for 5 days. The neurite outgrowth from PC12 cells in response this exposure confirmed the biological activity of NGF after encapsulation.

Wistar rats (200 to 250 g) were used for conduit implantation. After anaesthetizing with pentobarbital, the right sciatic nerve of the rat was exposed through a 3 cm long skin incision on the thigh and retraction of the gluteus maximus muscle. The nerve was freed from surrounding tissue and transected at the midthigh level, proximal to the tibial and peroneal bifurcation. A 5 to 7 mm piece of the nerve was removed and then the proximal 1 and distal nerve stumps were pulled 2 mm into each opening of the PPE tubes, leaving a 10-mm interstump gap. The two stumps were fixed within the tubes with a single 10-0 perineurial suture (Ethilon). Before the proximal stump was pulled into the tube opening, the tube was filled with NGF microspheres containing 100 μg of NGF. The surgery was performed under an Olympus operating microscope. The muscle layers were closed with 4/0 silk sutures and the skin closed with Michel clips.

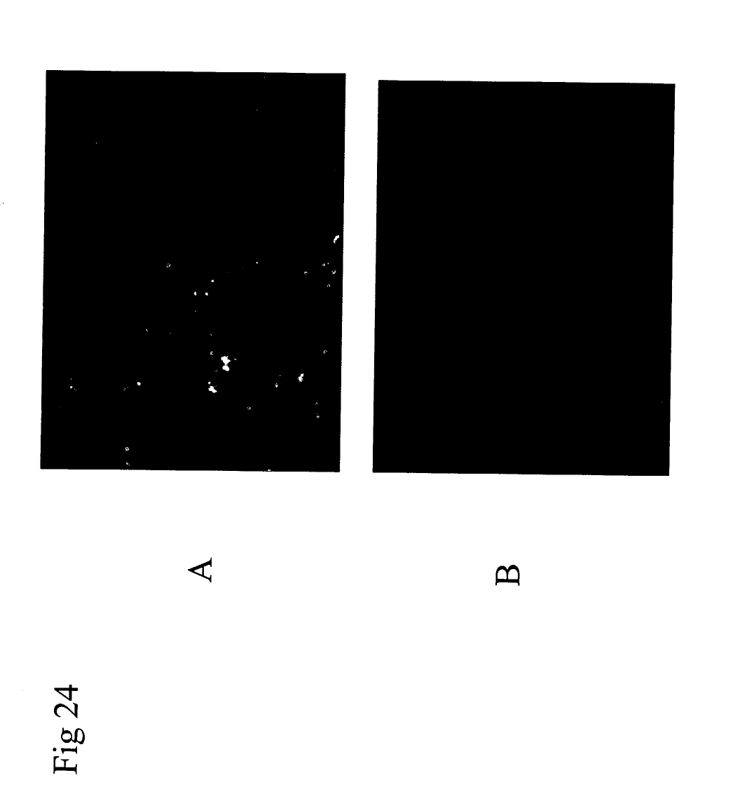

Two weeks after implantation, the conduits together with regenerated tissue samples were collected for analysis. Speed-up regeneration was observed in the conduits loaded with NGF microspheres, with many neurofilament-positive fibers at the distal stumps of truncated nerves (FIG. 24).

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosures of which are incorporated herein by reference.

References

1. Furnish E J, Schmidt C E. Tissue engineering of the peripheral nervous system. In: Patrick C W, Mikos A G, Mcintire L V, editor: Frontiers in tissue engineering. Elsevier Science Ltd, 1998:515-535.

2. Merle M, Dellon A L, Campbell J N, Chang P S, Complication from silicon-polymer intubulation of nerves. Microsurgery 1989;10:130-3.

3. Lundborg G, Rosen B, Abrahamson S O, Dahlin L, Danielsen N. Tubular repair of the median nerve in the human forearm. Preliminary findings. J Hand Surg 1994; 19B:273-6.

4. Borkenhagen M, Stoll R C, Neuenschwander P, Suter U W, Aebischer P, In vivo performance of a new biodegradable polyester urethane system used as a nerve guidance channel. Biomaterials 1998;19:2155-65.

5. Hoppen H J, Leenslag J W, Pennings A J, van der Lei B, Robinson PH. Two-ply biodegradable nerve guide: basic aspects of design, construction and biological performance. Biomaterials 1990; 11:286-90.

6. Robinson P H, van der Lei B, Hoppen H J, Leenslag J W, Pennings A J, Nieuwenhuis P. Nerve regeneration through a two-plybiodegradable nerve guide in the rat and the influence of ACTH-9 nerve growth factor. Microsurg 1991;12:412-9.

7. den Dunnen W F, Schakenraad J M, Zondervan G J, Pennings A J, van der Lei B, Robinson P H. A new PLLA/PCL copolymer for nerve regeneration. J Mater Sci Mater Med 1993;4:521-5.

8. den Dunnen W F, van der Lei B, Schakenraad J M, Blaauw E H, Stokroos I, Pennings A J, Robinson P H. Long term evaluation of nerve regeneration in a biodegradable nerve guide. Microsurg 1993; 14:508-15.

9. den Dunnen W F, van der Lei B, Robinson P H, Holwerda A, Pennings A J, Schakenraad J M, Biological performance of a degradable poly(lactic acid-epsilon- caprolactone) nerve guide: influence of tube dimensions. J Biomed Mater Res 1995;29: 757-66.

10. den Dunnen W F, Stokroos I, Blaauw E H, Holwerda A, Pennings A J, Robinson P H, Schakenraad J M. Light-microscopic and electron-microscopic evaluation of short-term nerve regeneration using a biodegradable poly(DL-latide-e-caprolactone) nerve guide. J Biomed Mater Res 1996; 31:105-15.

11. Perego G, Cella G D, Aldini N N, Fini M, Giardino R, Preparation of a new nerve guide from a poly(L-lactide-co-6-caprolactone). Biomaterials, 1994; 15:189-193.

12. Heath C A, and Rutkowski G E, The development of bioartificial nerve grafts for peripheral-nerve regeneration. TIBTECH, 1998;16:163-168.

13. Richards M, Dahiyat B I, Arm D M, Brown P R, Leong K W, Evaluation of polyphosphates and polyphosphonates as degradable biomaterials. J Biomed Mater Res, 1991;25:1151-67

14. Dahiyat B I, Hostin E, Posadas E M, Leong K W, Synthesis and characterization of putrescine-based poly(phosphoester-urethanes), J Biomater Sci Polym Ed, 1993;4:529-43.

15. Mao H-Q, Shtpanova-Kadiyaia I, Zhao Z, Dang W, Leong K W, Biodegrasable polymers: Poly(phosphoester)s. 45-60, Encyclopedia of controlled drop delivery. Ed. Mathiowitz E, Johnswiley & Sons, 1999, Inc. New York, N.Y.

16. Dahiyat B I, Richards M, Leong K W, Controlled release from poly(phophoester) matrices. J Controlled Release, 1995;33:13-21.

17. Lundborg G, Dahlin L, Dohi D, Kanje M, Terada N. A new type of “bioartificial” nerve graft for bridging extended defects in nerves. J Hand Surg 1997; 22B:299-303.

18. Williams, L. R., Exogenous fibrin matrix precursors stimulate the temporal progress of nerve regeneration within a silicone chamber. Neurochem Res. 1987;851-860.

19. Williams L R, Danielsen N, Muller H, Varon S. Influence of the acellular fibrin matrix on nrve regeneration success within a silicone chamber model.

In: The current status of peripheral nerve regeneration. Alan R. Liss, Inc. 1988: 111-122.

22. Wan, A A C, Mao H—Q, Wang S, Leong K W, Ong L K L L, Hanry Y.

Fabrication of poly(phosphoester) nerve guides by immersion precipitation and the control of porosity. Biomaterials, 2000, in press.

23. Guenard V, Kleitman N, Morrissey T K, Bunge R P, Aebischer P, Syngeneic Schwann cells derived from adult nerves seeded in semipermeable guidance channels enhance peripheral nerve regeneration. J. Neurosci. 1992;12:33 10-20.

24. Andriano K P, Tabata Y, Lkada Y, Heller J. In vitro and in vivo comparison of bulk and surface hydrolysis in absorbable polymer scaffolds for tissue engineering. J Biomed Mater Res 1999, 48:602-612.

25. Henry E W, Chiu T H, Nyilas E, Brushart T M, Dikkes P, Sidman R L, Nerve regeneration through biodegradable polyester tubes. Exp Neurol 1985;90:652-76.

26. Perego G, Vercellio T, Balbontin G, Copolymers of L and D,L-lactide with 6-caprolactone: synthesis and characterization. Macromol Chem 1993;

194:2463-2469.

27. Aebischer P, Salessiotis A N, Winn S R. Basic fibroblast growth factor released from synthetic guidance channels facilitates peripheral nerve regeneration across long nerve gaps. J. Neurosci Res 1989; 23:289-9.

What is claimed is:

1. A polymer comprising the subunit 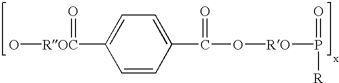
wherein R′ is ethyl or butyl and R and R″ are each a suitable side chain or a cross linking agent.

2. The polymer of claim 1, wherein R is O(CH₂)ₙCH₃ and n is between 1 and 12.

3. The polymer of claim 2, wherein R is OCH₂CH₃.

4. The polymer of any one of the preceding claims, wherein R′ is CH₂CH₂.

5. The polymer of any one of claims 1 to 3, wherein R″ is (CH₂)ₚ and p is between 1 and 12.

6. The polymer of claim 5, wherein R″ is CH₂CH₂.

7. The polymer of claim 1, wherein a bioactive component is conjugated to either or both of R and R″ via a functional group.

8. The polymer of claim 7, wherein the bioactive component comprises a neuroactive protein or peptide.

9. The polymer of claim 7, wherein the bioactive component comprises a neurotrophic factor.

10. The polymer of claim 9, wherein the neutrophic factor comprises NGF, BDNF, CNTF, or FGF.

11. The polymer of claim 8, wherein the bioactive component comprises a cell adhesive peptide.

12. The polymer of claim 11, wherein the cell adhesive peptide comprises the amino acids: Arg Gly Asp; Tyr Ile Gly Ser Arg; or Ile Lys Val Ala Val.

13. The polymer of claim 1, wherein either or both of R and R″ are a cross linking agent comprising one or more metal ions.

14. The polymer of claim 1, wherein either or both of R and R″ are a cross linking agent comprising one or more organic cross linking agents.

15. The polymer of claim 14, wherein the organic cross linking agent comprises 1,3,5-trihydroxybenzene or (CH₂OH)₄C.

16. The polymer of claim 1, further comprising the subunit

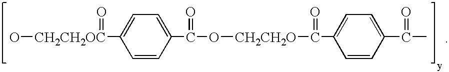

17. The polymer of claim 16, wherein x is between 5 and 100 and y is between 5 and 100.

18. The polymer of claim 17, wherein the ratio of x to y is 80:20.

19. The polymer of claim 16 further comprising the subunit

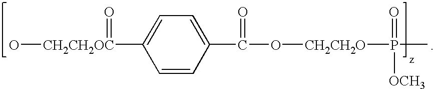

20. The polymer of claims 16 further comprising the subunit

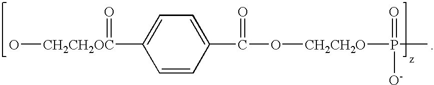

21. The polymer of claim 19 or 20, wherein x is between 5 and 100, y is between 5 and 100 and z is between 5 and 100.

22. A nerve guide conduit comprising a poly(phosphoester) polymer in the shape of a tube having a diameter, a first end, a second end, and a wall having an outer surface and a luminal surface.

23. A nerve guide conduit comprising a polymer according to claim 1.

24. The nerve guide conduit of claim 22, wherein the polymer has an average molecular weight of between 10,000 and 25,000.

25. The nerve guide conduit of claim 24, wherein the polymer has an average molecular weight of between 14,900 and 18,900.

26. The nerve guide conduit of claim 25, wherein the polymer has an average molecular weight of between 15,000 and 17,000.

27. The nerve guide conduit of claim 22, wherein the conduit has a surface porosity of between 2 and 58%.

28. The nerve guide conduit of claim 27, wherein the conduit has a surface porosity of 35%.

29. The nerve guide conduit of claim 27, wherein the conduit has a surface porosity of 8%.

30. The nerve guide conduit of claim 22, wherein the tube has a diameter of between 1 and 2 mm.

31. The nerve guide conduit of claim 30, wherein the diameter is 1.5 mm.

32. The nerve guide conduit of claims 22, wherein the wall has a thickness of between 150 and 250 mm.

33. The nerve guide conduit of claim 32, wherein the thickness is between 170 and 240 mm.

34. The nerve guide conduit of claim 22, wherein the wall comprises a plurality of layers.

35. The nerve guide conduit of claim 34 comprising at least 3 layers.

36. The nerve guide conduit of claim 34, wherein each layer is between 20 and 30 mm thick.

37. The nerve guide conduit of claim 36, wherein each layer is 25 mm thick.

38. The nerve guide conduit of claim 22, wherein the outer surface of the wall has greater microporosity than the luminal surface of the conduit.

39. The nerve guide conduit of claim 22 further comprising a gene delivery system.

40. The nerve guide conduit of claim 39, wherein the gene delivery system comprises a complex of DNA and a cationic polymer or lipid loaded into the conduit.

41. The nerve guide conduit of claim 40, wherein the complex is particles of 20 nm in diameter.

42. The nerve guide conduit of claim 40, wherein the cationic polymer or lipid comprises polyethylenimine, poly-L-lysine, or chitosan.

43. The nerve guide conduit of claim 40, wherein the cationic polymer or lipid comprises 1,2-dioleoyl phosphatidylethanolamine.

44. The nerve guide conduit of claim 43, wherein the cationic polymer or lipid comprises Transfast or GenePORTER.

45. The nerve guide conduit of any one of claims 39 to 44, wherein the gene encodes a neurotrophic protein or a neuro-active neural fibre growth eliciting molecule.

46. The nerve guide conduit of claim 45, wherein the gene comprises NGF, BDNF or Bcl-2.

47. The nerve guide conduit of claim 22, further comprising a sustained protein delivery system.

48. The nerve guide conduit of claim 47, wherein the sustained protein delivery system comprises one or more microspheres loaded into the conduit, wherein the microspheres contain a protein that is released from the microspheres progressively.

49. The nerve guide conduit of claim 48, wherein the microspheres are made from a poly(phosphoester) polymer.

50. The nerve guide conduit of claim 49, wherein the microspheres are made from a polymer comprising the subunit 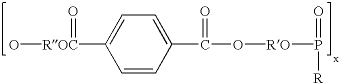
wherein R′ is ethyl or butyl and R and R″ are each a suitable side chain or a cross linking agent.

51. The nerve guide conduit of claim 48, wherein the microspheres are made from poly(lactic-co-glycolic acid) or poly(lactide-co-glycolide).

52. The nerve guide conduit of any one of claims 48 to 50, wherein the average diameter of the microspheres is between 5 and 20 mm.

53. The nerve guide conduit of claim 52, wherein the average diameter of the microspheres is 10 nm.

54. The nerve guide conduit of claim 48, wherein the microspheres release the protein over a period of at least three months.

55. The nerve guide conduit of claim 48, wherein at least 100 mm of protein is loaded per 10 mm of conduit.

56. The nerve guide conduit of claim 47, wherein the sustained protein delivery system comprises NGF, BDNF, CNTF, epidermal growth factor or fibroblast growth factor.

57. The nerve guide conduit of claim 22, wherein the conduit is loaded with a bioartificial nerve graft comprising Schwann cells.

58. A method of regenerating a severed nerve having first and second nerve stumps comprising the steps of:
providing a nerve guide conduit according to claim 22;
inserting the first nerve stump into the first end of the nerve conduit; and
inserting the second nerve stump into the second end of the nerve conduit.

59. The method of claim 58, wherein the nerve is in the hand and the conduit is provided adjacent the tendons of the hand.

60. A method of fabricating a polymer comprising the steps of:
providing a solution of the polymer and a solvent; and
adding a first non-solvent at a first concentration and second non-solvent at a second concentration to the solution to provide a mixture.

61. The method of claim 60, wherein the solubility of the polymer in the solvent is greater than 10 mg/ml.

62. The method of claim 61, wherein the solubility of the polymer in the solvent is greater than 100 mg/ml.

63. The method of any one of claims 60 to 62, wherein the solubility of the polymer in either or both of the non-solvents is less than 10 mg/ml.

64. The method of claim 63, wherein the solubility of the polymer in either or both of the non-solvents is less than 0.1 mg/ml.

65. The method of claim 60, wherein the solubility of the solvent in at least one of the first or second non-solvents or the solubility of at least one of the first or second non-solvents in the solvent is at least 0.5% w/w.

66. The method of claim 60 further comprising the step of increasing the concentration of the second non-solvent in the mixture.

67. The method of claim 66, wherein the concentration of the second non-solvent in the mixture is between 0 and 60%(v/v) and is increased to a concentration of between 65 and 75% (v/v).

68. The method of claim 67, wherein the concentration of the second non-solvent is increased to 70% (v/v).

69. The method according to claim 60, wherein the fabricated polymer has a predetermined porosity and further comprising the steps of, before adding the first and second solvents, determining the ratio of the first concentration to the second concentration required to achieve the predetermined porosity and then adding the first and second solvents in the required ratio.

70. The method of claim 69, wherein the step of determining the ratio of the first concentration to the second concentration comprises determining the demixing boundary and gelation point of the mixture.

71. The method of claim 70, wherein the step of determining the demixing boundary comprises titrating a pure non-solvent into the solution and detecting the concentration of non-solvent required to produce a permanent turbidity in the mixture.

72. The method of claim 70, wherein the step of determining the gelation point comprises evaporating the solvent from the solution, measuring the weight of the solution as a function of time to determine the rate of evaporation of the solvent, and detecting when the rate of evaporation shows a sharp decrease.

73. The method according of claim 60, wherein the solvent comprises chloroform.

74. The method of claims 60, wherein the first non-solvent comprises water.

75. The method of claims 60, wherein the second non-solvent comprises methanol.

76. The method of claim 60, wherein the polymer is a poly(phosphoester).

77. The method of claim 76, wherein the polymer is a poly(phosphoester) comprising the subunit 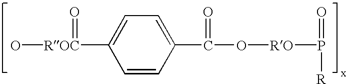
wherein R′ is ethyl or butyl and R and R″ are each a suitable side chain or a cross linking agent.

78. The method of claim 60 further comprising the steps of:
dipping a mandrel having a horizontal axis into the solution;
removing the mandrel from the solution to provide a coated mandrel; and
drying the solution on the coated mandrel.

79. A method of fabricating the nerve guide conduit of claim 22 comprising the steps of:
providing a solution comprising a polymer and a solvent;
dipping a mandrel having a horizontal axis into the solution;
removing the mandrel from the solution to provide a coated mandrel;
drying the solution on the coated mandrel to provide a polymer coated mandrel; and
removing the polymer from the polymer coated mandrel.

80. The method of any one of claim 78 or 79 further comprising the step of immersing the coated mandrel in a mixture comprising one or more non-solvents to provide a coated mandrel exposed to a non-solvent; and removing the one or more non-solvents from the coated mandrel exposed to a non-solvent.

81. The method of claim 80, wherein the steps of dipping the mandrel into the solution; removing the mandrel from the solution to provide the coated mandrel, and immersing the coated mandrel in the non-solvent are repeated one or more times to produce a mandrel with multiple polymer coatings.

82. The method according to claim 81, wherein the steps of dipping the mandrel into the solution, removing the mandrel from the solution to provide the coated mandrel, and immersing the coated mandrel in the non-solvent are repeated at least three times.

83. A method of claims 80, further comprising dipping the coated mandrel exposed to the non-solvent into the solution before the mixture of one or more non-solvents is completely removed from the coated mandrel exposed to a non-solvent.

84. The method according to any one of claim 78 or 79, wherein the drying is performed by freeze drying.

85. The method according to any one of claim 78 or 79, wherein the drying is performed by vacuum drying.

86. The method according to any one of claim 78 or 79 further comprising the step of rotating the coated mandrel along the horizontal axis to reduce variations in the thickness of the polymer on the polymer coated mandrel.

87. A method of fabricating a polymer comprising the steps of:
providing a solution comprising a polymer and a solvent;
casting the solution in a mold;
evaporating the solvent to provide a mold coated with the polymer; and
removing the polymer from the mold to provide the polymer as a sheet having first and second opposing edges.

88. A method of fabricating a nerve guide conduit according to claim 22 comprising the steps of:
providing a solution comprising a polymer and a solvent;
casting the solution in a mold;
evaporating the solvent to provide a mold coated with the polymer; and
removing the polymer from the mold to provide the polymer as a sheet having first and second opposing edges.

89. The method according to claim 87 or 88 further comprising the steps of rolling the polymer sheet around a mandrel so that the first and second opposing edges contact each other and sealing the first and second opposing edges together to form a tube.

90. The method according to claim 89, wherein the sealing is accomplished by exposing the contacted first and second opposing edges to a solvent.

91. The method according to claim 90, wherein the solvent is chloroform or dimethyl formamide.

92. The method of fabricating a nerve guide conduit according to claim 47 or 56 comprising the steps of:
mixing the protein delivery system with a solution or slurry of the poly(phosphoester) to provide a mixture; and
forming the mixture into the nerve guide conduit.

93. The method according to claim 92, wherein the solution or slurry is an aqueous solution or slurry.

94. The method of claim 79, wherein the solubility of the polymer in the solvent is greater than 10 mg/ml.

95. The method of claim 79, wherein the solubility of the polymer in the solvent is greater than 100 mg/ml.

96. The method of claim 80, wherein the solubility of the polymer in the one or more non-solvents is less than 10 mg/ml.

97. The method of claim 80, wherein the solubility of the polymer in the one or more non-solvents is less than 0.1 mg/ml.
